(19) United States
(12) Patent Application Publication
(10) Pub. No.: US 2022/0202680 A1
(43) Pub. Date: Jun. 30, 2022

(54) INCREASING THE STABILITY OF AGENTS FOR TREATING KERATIN MATERIAL

(71) Applicant: Henkel AG & Co. KGaA, Duesseldorf (DE)

(72) Inventors: Torsten LECHNER, Langenfeld (DE); Gabriele WESER, Neuss (DE); Claudia KOLONKO, Remscheid (DE); Caroline KRIENER, Duesseldorf (DE); Ulrike SCHUMACHER, Duesseldorf (DE); Marc NOWOTTNY, Monchengladbach (DE); Juergen SCHOEPGENS, Schwalmtal (DE); Phillip JAISER, Langenfeld (DE); Carsten MATHIASZYK, Essen (DE)

(73) Assignee: Henkel AG & Co. KGaA, Duesseldorf (DE)

(21) Appl. No.: 17/601,399

(22) PCT Filed: Feb. 17, 2020
(86) PCT No.: PCT/EP2020/054099

§ 371 Date: Oct. 4, 2021

(30) Foreign Application Priority Data
Apr. 4, 2019 (DE) 10 2019 204 808.0

(51) Int. Cl. A61K 8/58; A61K 8/06; A61K 8/894; A61K 8/81; A61Q 5/10

(52) U.S. Cl. CPC A61K 8/585; A61K 8/062; A61K 8/894; A61K 8/58; A61K 2800/95; A61Q 5/10; A61K 2800/884; A61K 2800/432; A61K 8/8147

(57) ABSTRACT

The object of the present disclosure is a method for treating keratinous material, in particular human hair, involving applying the following to the keratinous material
a first composition (A) comprising, relative to the total weight of the composition (A)
   (A1) less than 10% by weight of water and
   (A2) one or more organic C₁-C₆ alkoxy silanes and/or their condensation products, and
a second composition (B) comprising
   (B1) Water,
   (B2) at least a first surfactant, and
   (B3) at least a second surfactant which is structurally different from the first surfactant (B2).

### CROSS-REFERENCE TO RELATED APPLICATION

This application is a U.S. National-Stage entry under 35 U.S.C. § 371 based on International Application No. PCT/EP2020/054099, filed Feb. 17, 2020, which was published under PCT Article 21(2) and which claims priority to German Application No. 102019204808.0, filed Apr. 4, 2019, which are all hereby incorporated in their entirety by reference.

### TECHNICAL FIELD

The present application is in the field of cosmetics and concerns a process for the treatment of keratinous material, in particular human hair, which comprises the use of two compositions (A) and (B). Composition (A) is a low-water composition comprising at least one C₁-C₆ organic alkoxysilane, and composition (B) comprises, in addition to water, at least one first surfactant and at least one second surfactant, these two surfactants being structurally different from one another.

A second object of the present disclosure is a kit-of-parts for dyeing keratinous material, which comprises the two compositions (A) and (B) described above, separately packaged in two packaging units.

### BACKGROUND

Changing the shape and color of keratinous fibers, especially hair, is an important area of modern cosmetics. To change the hair color, the specialist knows various coloring systems depending on the coloring requirements. Oxidation dyes are usually used for permanent, intensive colorations with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the colorations obtained with direct dyes have a shorter shelf life and quicker wash ability. Dyeings with direct dyes usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

If the user wants particularly long-lasting colorations, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. It teaches that by using a combination of pigment, organic silicon compound, hydrophobic polymer and a solvent, it is possible to create colorations on hair that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these alkoxy silanes or their hydrolysis or condensation products are applied to keratinous material, a film or coating is formed on the keratinous material which completely envelops the keratinous material and in this way strongly influences the properties of the keratinous material. Possible areas of application include permanent styling or permanent shape modification of keratin fibers. In this process, the keratin fibers are mechanically shaped into the desired form and then fixed in this form by forming the coating described above. Another particularly suitable application is the coloring of keratin material. In this application, the coating or film is produced in the presence of a coloring compound, for example a pigment. The film colored by the pigment remains on the keratin material or the keratin fibers and results in surprisingly wash-resistant dyeing.

The great advantage of the alkoxy-silane based dyeing principle is that the high reactivity of this class of compounds allows a very fast coating. This means that extremely good dyeing results can be achieved after very short application periods of only a few minutes. In addition to these advantages, however, the high reactivity of alkoxy silanes also has some disadvantages.

Due to their high level of reactivity, the organic alkoxy silanes cannot be prepared together with larger amounts of water, since a large excess of water initiates immediate hydrolysis and subsequent polymerization. The polymerization that takes place during storage of the alkoxy silanes in aqueous medium manifests itself in a thickening or gelation of the aqueous preparation. This makes the preparations so highly viscous and gelatinous that they can no longer be applied evenly to the keratin material. In addition, storage of the alkoxy silanes in the presence of high amounts of water is associated with a loss of their reactivity, so that the formation of a resistant coating on the keratin material is also no longer possible.

### BRIEF SUMMARY

Methods for treating keratinous material, and kits-of-parts for the same, are provided. In an exemplary embodiment, a method includes applying a first composition (A) and a second composition (B) to the keratinous material. The first composition (A) comprises less than about 10 weight % water and a C₁-C₆ alkoxy silane and/or a condensation product of the alkoxy silane. The second composition (B) comprises water, a first surfactant (B2), and a second surfactant (B3) that is structurally different than the first surfactant (B2).

A kit-of-parts is provided in another embodiment. The kit of parts includes a first container containing a first composition (A) and a second container containing a second composition (B). The first composition (A) comprises less than about 10 weight % water and a C₁-C₆ alkoxy silane and/or a condensation product of the alkoxy silane. The second composition (B) comprises water, a first surfactant (B2), and a second surfactant (B3) that is structurally different than the first surfactant (B2).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

For these reasons, it is necessary to store the organic alkoxy silanes in an anhydrous or anhydrous environment and to prepare the corresponding preparations in a separate container. Due to their high level of reactivity, alkoxy silanes can react not only with water but also with other cosmetic ingredients. In order to avoid all undesirable reactions, the preparations containing alkoxy silanes therefore preferably do not contain any other ingredients or contain only those selected ingredients which have proved to be chemically inert to the alkoxy silanes. Accordingly, the concentration of alkoxy silanes in the preparation is preferably chosen to be relatively high. The low-water preparations containing the alkoxy silanes in relatively high concentrations can also be referred to as “silane blends”.

For application to the keratin material, the user must now convert this relatively highly concentrated silane blend into a ready-to-use mixture. In this ready-to-use mixture, on the one hand the concentration of organic alkoxy silanes is reduced, and on the other hand the application mixture also contains a higher proportion of water (or an alternative ingredient), which triggers the polymerization leading to the coating.

It has proved to be an extremely great challenge to optimally adapt the polymerization rate, i.e., the speed at which the coating forms on the keratin material, to the application conditions.

When applied to human hair, for example, a polymerization rate that is too fast will result in polymerization being completed before all sections of hair have been treated. Therefore, too fast polymerization makes the whole-head treatment impossible. In the dyeing process, the excessively fast polymerization manifests itself in an extremely uneven color result, so that the hair sections that were treated last are only poorly colored.

On the other hand, if polymerization is too slow, all areas of the hair can be treated without time pressure, but this increases the application time. Therefore, if polymerization is too slow, the great advantage of this dyeing technology, the formation of washfast colorations within shortest application periods, does not come into effect.

The object of the present application was to find a process for treating keratinous material by controlling the rate of polymerization of organic alkoxy-silanes could be adapted to the conditions of use, in particular to the conditions prevailing when applied to the human head. In other words, a process was sought by which the organic alkoxy-silanes would remain reactive long enough to permit whole-head treatment without unduly prolonging the application period.

Surprisingly, it has been found that this task can be fully solved if the keratin material is treated in a process in which two compositions (A) and (B) are applied to the keratin material. The first composition (A) is the low water silane blend described previously. The second composition (B) is water-containing and also contains at least two structurally different surfactants. During application, both compositions (A) and (B) come into contact with each other, whereby this contact can be made either by prior mixing of (A) and (B) or by successive application of (A) and (B) to the keratin material.

A first object of the present disclosure is a method for treating keratinous material, in particular human hair, involving applying the following to the keratinous material
a first composition (A) comprising, relative to the total weight of the composition (A)
   (A1) less than about 10% by weight of water and
   (A2) one or more organic C₁-C₆ alkoxy silanes and/or their condensation products, and
a second composition (B) comprising
   (B1) Water,
   (B2) at least a first surfactant, and
   (B3) at least a second surfactant which is structurally different from the first surfactant (B2).

A first object of the present disclosure is a method for treating keratinous material, in particular human hair, involving applying the following to the keratinous material
a first composition (A) comprising, relative to the total weight of the composition (A)
   (A1) less than about 10% by weight of water and
   (A2) one or more organic C₁-C₆ alkoxy silanes, and
a second composition (B) comprising
   (B1) Water,
   (B2) at least a first surfactant, and
   (B3) at least a second surfactant which is structurally different from the first surfactant (B2).

It has been shown that the surfactants (B2) and (B3) contained in the water-containing composition (B) reduce the polymerization rate of the organic C₁-C₆ alkoxy silanes (A2) upon contact with the composition (A). Surprisingly, the reactivity of the organic C₁-C₆ alkoxy silanes (A2) could thus be optimally adapted to the application conditions prevailing in a whole-head hair dyeing process. Even more complicated or time-consuming dyeing techniques, such as the dyeing of highlights specially arranged on the head, could be realized by using the method according to the present disclosure. When the two compositions (A) and (B) were used in a dyeing process on keratinous material, in particular on human hair, it was possible in this way to obtain colorations with a particularly high degree of uniformity.

### Treatment of Keratinous Material

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair in particular.

Agents for treating keratinous material are understood to mean, for example, agents for coloring the keratinous material, agents for reshaping or shaping keratinous material, in particular keratinous fibers, or agents for conditioning or caring for the keratinous material. The agents prepared by the process according to the present disclosure are particularly suitable for dyeing keratinous material, in particular for dyeing keratinous fibers, which are preferably human hair.

The term “coloring agent” is used in the context of the present disclosure to refer to a coloring of the keratin material, in particular of the hair, caused by the use of coloring compounds, such as thermochromic and photochromic dyes, pigments, mica, direct dyes and/or oxidation dyes. In this staining process, the aforementioned colorant compounds are deposited in a particularly homogeneous and smooth film on the surface of the keratin material or diffuse into the keratin fiber. The film is formed in situ by oligomerization or polymerization of the organic alkoxy silane(s), and by the interaction of the colorant compound and organic silicon compound and optionally other components, such as a film-forming polymer.

### Water Content (A1) in the Composition (A)

The process according to the present disclosure is exemplified by the application of a first composition (A) to the keratinous material.

To ensure a sufficiently high storage stability, composition (A) is exemplified in that it is low in water, preferably substantially free of water. Therefore, the composition (A) contains less than about 10% by weight of water, based on the total weight of the composition (A).

With a water content of just under about 10% by weight, the compositions (A) are stable in storage over long periods. However, in order to further improve the storage stability and to ensure a sufficiently high reactivity of the organic C₁-C₆ alkoxy silanes (A2), it has been found to be particularly preferable to further lower the water content in the composition (A). For this reason, first composition (A) preferably contains about 0.01 to about 9.5% by weight, more preferably about 0.01 to about 8.0% by weight, still more preferably about 0.01 to about 6.0 and most preferably about 0.01 to about 4.0% by weight of water (A1), based on the total weight of composition (A).

In one particularly preferred version, a process according to the present disclosure is exemplified in that the first composition (A) contains about 0.01 to about 9.5% by weight, preferably about 0.01 to about 8.0% by weight, more preferably about 0.01 to about 6.0 and most preferably about 0.01 to about 4.0% by weight of water (A1), based on the total weight of the composition (A).

Organic C₁-C₆ Alkoxy Silanes (A2) and/or their Condensation Products in the Composition (A)

The composition (A) is exemplified in that it comprises one or more organic C₁-C₆ alkoxy silanes (A2) and/or their condensation products.

The organic C₁-C₆ alkoxy silane(s) are organic, non-polymeric silicon compounds, preferably selected from the group of silanes containing one, two or three silicon atoms.

Organic silicon compounds, alternatively known as organosilicon compounds, are compounds that either have a direct silicon-carbon (Si—C) bond or in which the carbon is attached to the silicon atom via an oxygen, nitrogen or sulfur atom. The organic silicon compounds according to the present disclosure are preferably compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups.

Typically, the C₁-C₆ alkoxy silanes of the present disclosure have at least one C₁-C₆ alkoxy group bonded directly to a silicon atom. The C₁-C₆ alkoxy silanes according to the present disclosure thus comprise at least one structural unit R′R″R′″Si—O—(C₁-C₆ alkyl) where the radicals R′, R″ and R′″ represent the three remaining bond valencies of the silicon atom.

The C₁-C₆ alkoxy group or groups bonded to the silicon atom are very reactive and are hydrolyzed at high rates in the presence of water, the rate of reaction depending, among other things, on the number of hydrolyzable groups per molecule. If the hydrolyzable C₁-C₆ alkoxy group is an ethoxy group, the organic silicon compound preferably contains a structural unit R′R″R″Si—O—CH₂—CH₃. The residues R′, R″ and R′″ again represent the three remaining free valences of the silicon atom.

Even the addition of small amounts of water leads first to hydrolysis and then to a condensation reaction between the organic alkoxy silanes. For this reason, both the organic alkoxy silanes (A2) and their condensation products may be present in the composition.

A condensation product is understood to be a product formed by the reaction of at least two organic C₁-C₆ alkoxy silanes with elimination of water and/or with elimination of a C₁-C₆ alkanol.

The condensation products can be, for example, dimers, but also trimers or oligomers, the condensation products being in equilibrium with the monomers.

Depending on the amount of water used or consumed in the hydrolysis, the equilibrium shifts from monomeric C₁-C₆ alkoxysilane to condensation product.

In a highly preferred version, a process according to the present disclosure is exemplified in that the composition (A) comprises one or more organic C₁-C₆ alkoxy silanes (A2) selected from silanes having one, two or three silicon atoms, the organic silicon compound further comprising one or more basic chemical functions.

This basic group can be, for example, an amino group, an alkylamino group or a dialkylamino group, which is preferably connected to a silicon atom via a linker. Preferably, the basic group is an amino group, a C₁-C₆ alkylamino group or a di(C₁-C₆)alkylamino group.

A highly preferred method according to the present disclosure is exemplified in that the composition (A) comprises one or more organic C₁-C₆ alkoxy silanes (A2) selected from the group of silanes having one, two or three silicon atoms, and wherein the C₁-C₆ alkoxy silanes further comprise one or more basic chemical functions.

Particularly good results were obtained when C₁-C₆ alkoxy silanes of formula (S-I) and/or (S-II) were used in the process according to the present disclosure. Since, as previously described, hydrolysis/condensation already starts at trace amounts of moisture, the condensation products of the C₁-C₆ alkoxy silanes of formula (S-I) and/or (S-II) are also encompassed by this version.

In another highly preferred version, a process according to the present disclosure is exemplified in that the first composition (A) comprises one or more organic C₁-C₆ alkoxy silanes (A2) of the formula (S-I) and/or (S-II),

R₁R₂N-L-Si(OR₃)ₐ(R₄)_{b} (S4)

where
R₁, R₂ independently represent a hydrogen atom or a C₁-C₆ alkyl group,
L is a linear or branched divalent C₁-C₂₀ alkylene group,
R₃, R₄ independently represent a C₁-C₆ alkyl group,
a, stands for an integer from 1 to 3, and
b is the integer 3-a, and

(R₅O)_{c}(R₆)_{d}Si-(A)ₑ-[NR₇-(A′)]_{f}—[O-(A″)]_{g}—[NR₈-(A′″)]ₕ—Si(R_{6′})_{d′}(OR_{5′})_{c′} (S-II),

where
R₅, R_{5′}, R_{5″}, R₆, R_{6′} and R_{6″} independently represent a C₁-C₆ alkyl group,
A, A′, A″, A′ and A″ independently represent a linear or branched C₁-C₂₀ divalent alkylene group,
R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (S-III),

-(A″″)-Si(R_{6″})_{d″}(OR_{5″})_{c″} (S-III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0,

and/or their condensation products.

The substituents R₁, R₂, R₃, R₄, R₅, R_{5′}, R_{5″}, R₆, R_{6′}, R_{6″}, R₇, R₈, L, A, A′, A″, A′″ and A″″ in the compounds of formula (S-I) and (S-II) are exemplified below: Examples of a C₁-C₆ alkyl group include methyl, ethyl, propyl, isopropyl, n-butyl, s-butyl and t-butyl, n-pentyl and n-hexyl groups. Propyl, ethyl and methyl are preferred alkyl radicals. Examples of a C₂-C₆ alkenyl group include vinyl, allyl, but-2-enyl, but-3-enyl, and isobutenyl; preferred C₂-C₆ alkenyl radicals include vinyl and allyl. Preferred examples of a hydroxy-C₁-C₆-alkyl group include a hydroxymethyl, a 2-hydroxyethyl, a 2-hydroxypropyl, a 3-hydroxypropyl, a 4-hydroxybutyl, a 5-hydroxypentyl and a 6-hydroxyhexyl group; a 2-hydroxyethyl group is particularly preferred. Examples of an amino-C₁-C₆-alkyl group include the aminomethyl group, the 2-aminoethyl group, the 3-aminopropyl group. The 2-aminoethyl group is particularly preferred. Examples of a linear divalent C₁-C₂₀ alkylene group include, for example, the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched C₃-C₂₀ divalent alkylene groups include (—CH₂—CH(CH₃)—) and (—CH₂—CH(CH₃)—CH₂—).

In the organic silicon compounds of the formula (S-I)

R₁R₂N-L-Si(OR₃)ₐ(R₄)_{b} (S-I),

R₁ and R₂ independently represent a hydrogen atom or a C₁-C₆ alkyl group. Most preferably, R₁ and R2 are both hydrogen atom.

In the middle part of the organic silicon compound is the structural unit or linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each -L- grouping may form two bonds.

Preferably, -L- represents a linear, divalent C₁-C₂₀ alkylene group. More preferred would be if -L- represents a linear divalent C₁-C₆ alkylene group. Particularly preferred would be if -L- represents a methylene group (—CH₂—), an ethylene group (—CH₂—CH₂—), a propylene group (—CH₂—CH₂—CH₂—) or a butylene group (—CH₂—CH₂—CH₂—CH₂—). Extremely preferred would be if L represents a propylene group (—CH₂—CH₂—CH₂—).

The organic silicon compounds according to the present disclosure of the formula (S-I)

R₁R₂N-L-Si(OR₃)ₐ(R₄)_{b} (S-I),

each carry at one end the silicon-containing grouping —Si(OR₃)ₐ(R₄)_{b}.

In the terminal structural unit —Si(OR₃)ₐ(R₄)_{b}, R₃ and R₄ independently represent a C₁-C₆ alkyl group, particularly preferably R₃ and R₄ independently represent a methyl group or an ethyl group.

In this case, a stands for an integer from 1 to 3, and b stands for the integer 3-a. If a stands for the number 3, then b is equal to 0. If a stands for the number 2, then b is equal to 1. If a stands for the number 1, then b is equal to 2.

Keratin treatment agents with particularly good properties could be prepared if the composition (A) contains at least one organic C₁-C₆ alkoxy silane of the formula (S-I) in which the radicals R₃, R₄ independently of one another represent a methyl group or an ethyl group.

Furthermore, colorations with the best wash fastnesses could be obtained if the composition (A) contains at least one organic C₁-C₆ alkoxy silane of the formula (S-I) in which the radical a represents the number 3. In this case the rest b stands for the number 0.

In another preferred version, a process according to the present disclosure is exemplified in that the composition (A) comprises one or more organic C₁-C₆ alkoxy silanes of formula (S-I),

where
R₃, R₄ independently represent a methyl group or an ethyl group, and
a stands for the number 3 and
b stands for the number 0.

In another preferred version, a process according to the present disclosure is exemplified in that the composition (A) comprises at least one or more organic C₁-C₆ alkoxy silanes of formula (S-I),

R₁R₂N-L-Si(OR₃)ₐ(R₄)_{b} (S-I),

where
R₁, R₂ both represent a hydrogen atom, and
L is a linear, divalent C₁-C₆ alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a stands for the number 3 and
b stands for the number 0.

Organic silicon compounds of the formula (I) which are particularly suitable for solving the problem according to the present disclosure are
(3-Aminopropyl)triethoxysilane

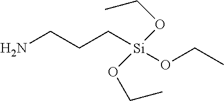

(3-Aminopropyl)trimethoxysilane

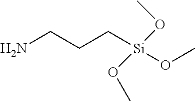

(2-Aminoethyl)triethoxysilane

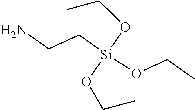

(2-Aminoethyl)trimethoxysilane

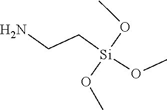

(3-Dimethylaminopropyl)triethoxysilane

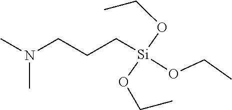 [SMILES: CCO[Si](CCCN(C)C)(OCC)OCC]

(3-Dimethylaminopropyl)trimethoxysilane

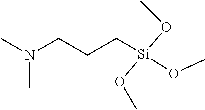

(2-Dimethylaminoethyl)triethoxysilane.

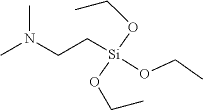

(2-Dimethylaminoethyl)trimethoxysilane and/or

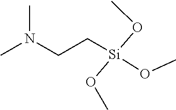

In a further preferred version, a process according to the present disclosure is exemplified in that the first composition (A) comprises at least one C₁-C₆ organic alkoxysilane (A2) of formula (S-I) selected from the group of
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-Dimethylaminoethyl)triethoxysilane,
(2-Dimethylaminoethyl)trimethoxysilane
   and/or their condensation products.

The aforementioned organic silicon compound of formula (I) is commercially available. (3-aminopropyl)trimethoxysilane, for example, can be purchased from SIGMA-ALDRICH®. (3-aminopropyl)triethoxysilane is also commercially available from SIGMA-ALDRICH®.

In another version of the method according to the present disclosure, the composition (A) may also comprise one or more organic C₁-C₆ alkoxy silanes of formula (S-II),

(R₅O)_{c}(R₆)_{d}Si-(A)ₑ-[NR₇-(A′)]_{f}—[O-(A″)]_{g}—[NR₈-(A′″)]ₕ-Si(R_{6′})_{d′}(OR_{5′})_{c′} (S-II).

The organosilicon compounds of the formula (S-II) according to the present disclosure each bear at their two ends the silicon-containing groupings (R₅O)_{c}(R₆)_{d}Si— and —Si(R_{6′})_{d′}(OR_{5′})_{c′}.

In the middle part of the molecule of formula (S-II) there are the groupings -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) according to the present disclosure comprises at least one grouping selected from the group of -(A)- and —[NR₇-(A′)]- and [O-(A″)]- and —[NR₈-(A′″)]-.

In the two terminal structural units (R₅O)_{c}(R₆)_{d}Si— and —Si(R_{6′})_{d′}(OR_{5′})_{c′}, the residues R₅, R_{5′}, R_{5″} independently represent a C₁-C₆ alkyl group. The R₆, R_{6′} and R_{6″} residues independently represent a C₁-C₆ alkyl group.

Here c stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is equal to 1. If c′ stands for the number 1, then d′ is 2.

Colorations with the best wash fastness values could be obtained if the residues c and c′ both stand for the number 3. In this case d and d′ both stand for the number 0.

In another preferred version, a process according to the present disclosure is exemplified in that the composition (A) comprises one or more organic C₁-C₆ alkoxy silanes of formula (S-II),

(R₅O)_{c}(R₆)_{d}Si-(A)ₑ-[NR₇-(A′)]_{f}—[O-(A″)]_{g}—[NR₈-(A′″)]ₕ-Si(R_{6′})_{d′}(OR_{5′})_{c′} (S-II),

where
R₅ and R_{5′} independently represent a methyl group or an ethyl group,
c and c′ both stand for the number 3 and
d and d′ both stand for the number 0.

When c and c′ are both 3 and d and d′ are both 0, the organic silicon compounds according to the present disclosure correspond to the formula (S-IIa)

(R₅O)₃Si-(A)ₑ-[NR₇-(A′)]_{f}[O-(A″)]_{g}[NR₈-(A′″)]ₕ—Si(OR_{5′})₃ (S-IIa).

The radicals e, f, g and h may independently represent the number 0 or 1, with at least one of e, f, g and h being different from zero. The abbreviations e, f, g and h thus define which of the groupings -(A)ₑ- and [NR₇-(A′)]_{f}- and [O-(A″)]_{g}- and —[NR₈-(A′″)]ₕ- are in the middle part of the organic silicon compound of the formula (II).

In this context, the presence of certain groupings has proven to be particularly advantageous in terms of achieving washable dyeing results. Particularly good results were obtained when at least two of the residues e, f, g and h stand for the number 1. Especially preferred e and f both stand for the number 1. Furthermore, g and h both stand for the number 0.

When e and f are both 1 and g and h are both 0, the organic silicon compounds according to the present disclosure correspond to the formula (S-IIb)

(R₅O)_{c}(R₆)_{d}Si-(A)-[NR₇-(A′)]-Si(R_{6′})_{d′}(OR_{5′})_{c′} (S-IIb).

A, A′, A″, A′″ and A″″ independently represent a linear or branched C₁-C₂₀ divalent alkylene group. Preferably, A, A′, A″, A′″ and A″″ independently represent a linear divalent C₁-C₂₀ alkylene group. Further preferably, A, A′, A″, A′″ and A″″ independently represent a linear divalent C₁-C₆ alkylene group.

The divalent C₁-C₂₀ alkylene group may alternatively be referred to as a divalent C₁-C₂₀ alkylene group, by which is meant that each grouping A, A′, A″, A′″ and A″″ may form two bonds.

Particularly preferred would be if A, A′, A″, A′″ and A″″ independently represent a methylene group (—CH₂—), an ethylene group (—CH₂—CH₂—), a propylene group (—CH₂—CH₂—CH₂—) or a butylene group (—CH₂—CH₂—CH₂—CH₂—). It would be extremely preferred if the radicals A, A′, A″, A′″ and A″″ represent a propylene group (—CH₂—CH₂—CH₂—).

When the radical f represents the number 1, the organic silicon compound of formula (II) according to the present disclosure contains a structural grouping —[NR₇-(A′)]-. When the radical h represents the number 1, the organic silicon compound of formula (II) according to the present disclosure contains a structural grouping —[NR₈-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of formula (S-III)

-(A″″)-Si(R_{6″})_{d″}(OR_{5″})_{c″} (S-III).

Very much preferred, R₇ and R₈ independently represent a hydrogen atom, a methyl group, a 2-hydroxyethyl group, a 2-alkenyl group, a 2-aminoethyl group or a group of formula (S-III).

When the radical f represents the number 1 and the radical h represents the number 0, the organic silicone compound according to the present disclosure contains the grouping [NR₇-(A′)], but does not contain the grouping —[NR₈-(A′″)]. If the radical R₇ now stands for a grouping of the formula (III), the organic silicone compound comprises 3 reactive silane groups.

In another preferred version, a process according to the present disclosure is exemplified in that the composition (A) comprises one or more organic C₁-C₆ alkoxy silanes (A2) of formula (S-II)

(R₅O)_{c}(R₆)_{d}Si-(A)ₑ-[NR₇-(A′)]_{f}—[O-(A″)]_{g}-[NR₈-(A′″)]ₕ-Si(R_{6′})_{d′}(OR_{5′})_{c′} (II),

where
e and f both stand for the number 1,
g and h both stand for the number 0,
A and A′ independently represent a linear divalent C₁-C₆ alkylene group
   and
R₇ represents a hydrogen atom, a methyl group, a 2-hydroxyethyl group, a 2-alkenyl group, a 2-aminoethyl group or a group of the formula (S-III).

In a further preferred version, a process according to the present disclosure is exemplified in that the composition (A) comprises one or more organic C₁-C₆ alkoxy silanes (A2) of formula (S-II), wherein
e and f both stand for the number 1,
g and h both stand for the number 0,
A and A′ independently represent a methylene group (—CH₂—), an ethylene group (—CH₂—CH₂—) or a propylene group (—CH₂—CH₂—CH₂),
   and
R₇ represents a hydrogen atom, a methyl group, a 2-hydroxyethyl group, a 2-alkenyl group, a 2-aminoethyl group or a group of the formula (S-III).

Organic silicon compounds of the formula (S-II) which are well suited for solving the problem according to the present disclosure are
3-(trimethoxysilyl)-N-[3-(trimethoxysilyl)propyl]-1-propanamine

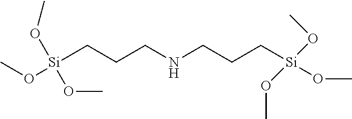

3-(Triethoxysilyl)-N-[3-(triethoxysilyl)propyl]-1-propanamine

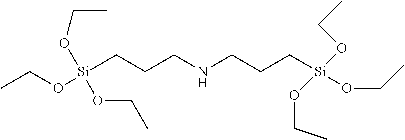 [SMILES: CCO[Si](CCCNCCC[Si](OCC)(OCC)OCC)(OCC)OCC]

N-methyl-3-(trimethoxysilyl)-N-[3-(trimethoxysilyl)propyl]-1-propanamine

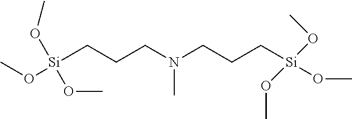

N-Methyl-3-(triethoxysilyl)-N-[3-(triethoxysilyl)propyl]-1-propanamine

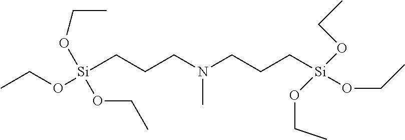 [SMILES: CCO[Si](CCCN(C)CCC[Si](OCC)(OCC)OCC)(OCC)OCC]

2-[Bis[3-(trimethoxysilyl)propyl]amino]-ethanol

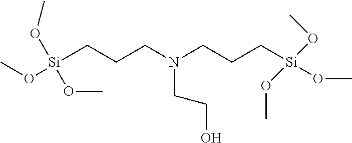

2-[bis[3-(triethoxysilyl)propyl]amino]ethanol

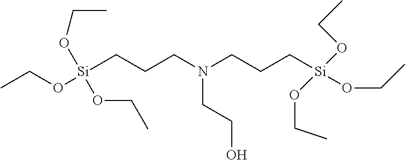 [SMILES: CCO[Si](CCCN(CCO)CCC[Si](OCC)(OCC)OCC)(OCC)OCC]

3-(Trimethoxysilyl)-N,N-bis[3-(trimethoxysilyl)propyl]-1-propanamine

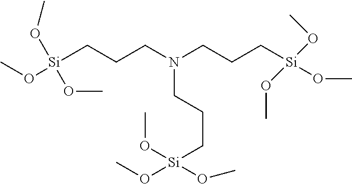

3-(Triethoxysilyl)-N,N-bis[3-(triethoxysilyl)propyl]-1-propanamine

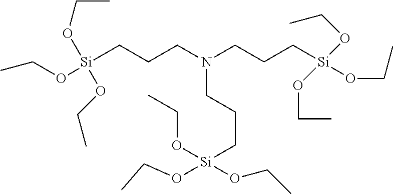 [SMILES: CCO[Si](CCCN(CCC[Si](OCC)(OCC)OCC)CCC[Si](OCC)(OCC)OCC)(OCC)OCC]

N1,N1-Bis[3-(trimethoxysilyl)propyl]-1,2-ethanediamine,

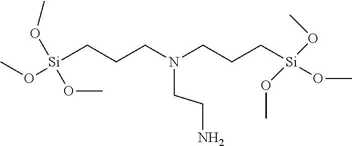

N1,N1-Bis[3-(triethoxysilyl)propyl]-1,2-ethanediamine,

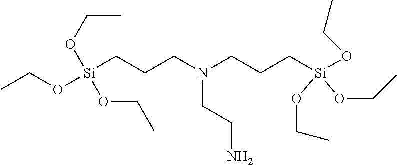 [SMILES: CCO[Si](CCCN(CCN)CCC[Si](OCC)(OCC)OCC)(OCC)OCC]

N,N-Bis[3-(trimethoxysilyl)propyl]-2-propen-1-amine

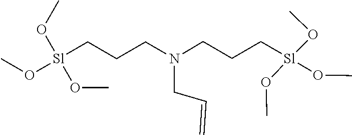

N,N-Bis[3-(triethoxysilyl)propyl]-2-propen-1-amine

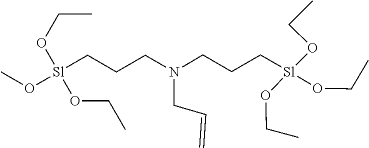

The aforementioned organic silicon compounds of formula (S-II) are commercially available.

Bis(trimethoxysilylpropyl)amines with the CAS number 82985-35-1 can be purchased from SIGMA-ALDRICH®.
Bis[3-(triethoxysilyl)propyl]amines with the CAS number 13497-18-2 can be purchased from SIGMA-ALDRICH®, for example.
N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from SIGMA-ALDRICH® or FLUOROCHEM®.
3-(triethoxysilyl)-N,N-bis[3-(triethoxysilyl)propyl]-1-propanamine with the CAS number 18784-74-2 can be purchased for example from FLUOROCHEM® or SIGMA-ALDRICH®.

In another preferred version, a process according to the present disclosure is exemplified in that the composition (A) comprises one or more organic C₁-C₆ alkoxy silanes of formula (S-II) selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol 2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.
   and/or their condensation products.

In further dyeing experiments, it has also been found to be quite particularly advantageous if at least one organic C₁-C₆ alkoxy silane (A2) of the formula (S-IV) was used in the process according to the present disclosure.

R₉Si(OR₁₀)ₖ(R₁₁)ₘ (S-IV).

The compounds of formula (S-IV) are organic silicon compounds selected from silanes having one, two or three silicon atoms, wherein the organic silicon compound comprises one or more hydrolysable groups per molecule.

The organic silicon compound(s) of formula (S-IV) may also be referred to as silanes of the alkyl-C₁-C₆-alkoxy-silane type,

R₉Si(OR₁₀)ₖ(R₁₁)ₘ (S-IV),

where
R₉ represents a C₁-C₁₂ alkyl group,
R₁₀ stands for a C₁-C₆ alkyl group,
R₁₁ stands for a C₁-C₆ alkyl group
k is an integer from 1 to 3, and
m stands for the integer 3-k.

In a further version, a particularly preferred method according to the present disclosure is exemplified in that the first composition (A) comprises one or more organic C₁-C₆ alkoxy silanes (A2) of the formula (S-IV),

R₉Si(OR₁₀)ₖ(R₁₁)ₘ (S-IV),

where
R₉ represents a C₁-C₁₂ alkyl group,
R₁₀ stands for a C₁-C₆ alkyl group,
R₁₁ stands for a C₁-C₆ alkyl group
k is an integer from 1 to 3, and
m stands for the integer 3-k.

and/or their condensation products.

In the organic C₁-C₆ alkoxy silanes of formula (S-IV), the R9 radical represents a C₁-C₁₂ alkyl group. This C₁-C₁₂ alkyl group is saturated and can be linear or branched. Preferably, R₉ represents a linear C₁-C₈ alkyl group. Preferably, R₉ represents a methyl group, an ethyl group, an n-propyl group, an n-butyl group, an n-pentyl group, an n-hexyl group, an n-octyl group, or an n-dodecyl group. Especially preferred, R₉ represents a methyl group, an ethyl group or an n-octyl group.

In the organic silicon compounds of formula (S-IV), the radical R₁₀ represents a C₁-C₆ alkyl group. Especially preferred, R₁₀ stands for a methyl group or an ethyl group.

In the organic silicon compounds of the formula (S-IV), the radical R₁₁ represents a C₁-C₆ alkyl group. In particular, Ru stands for a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Colorations with the best wash fastnesses were obtained when the composition (A) contains at least one organic C₁-C₆ alkoxy silane (A2) of the formula (S-IV) in which the radical k represents the number 3. In this case the rest m stands for the number 0.

Organic silicon compounds of the formula (S-IV) which are particularly suitable for solving the problem according to the present disclosure are
Methyltrimethoxysilane

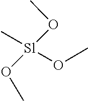

Methyltriethoxysilane

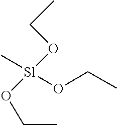

Ethyltrimethoxysilane

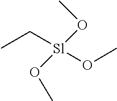

Ethyltriethoxysilane

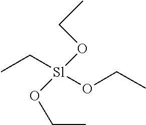

n-Propyltrimethoxysilane (also known as propyltrimethoxysilane)

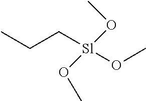

n-Propyltriethoxysilane (also known as propyltriethoxysilane)

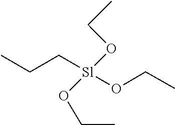

n-Hexyltrimethoxysilane (also called hexyltrimethoxysilane)

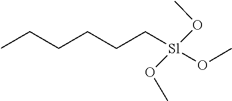

n-Hexyltriethoxysilane (also called hexyltriethoxysilane)

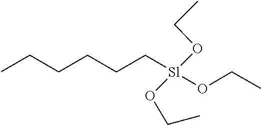

n-Octyltrimethoxysilane (also known as octyltrimethoxysilane)

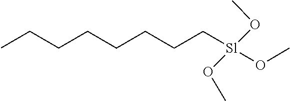

n-Octyltriethoxysilane (also known as octyltriethoxysilane)

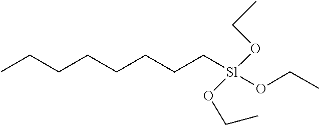

n-Dodecyltrimethoxysilane (also called dodecyltrimethoxysilane) and/or

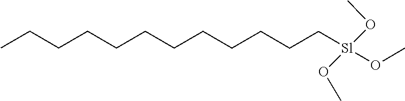

n-Dodecyltriethoxysilane (also referred to as dodecyltriethoxysilane).

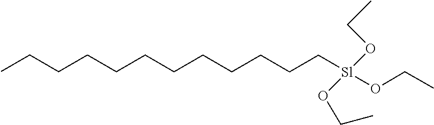

In a further preferred version, a process according to the present disclosure is exemplified in that the first composition (A) comprises at least one C₁-C₆ organic alkoxysilane (A2) of formula (S-IV) selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane.

and/or their condensation products.

The corresponding hydrolysis or condensation products are, for example, the following compounds:

hydrolysis of C₁-C₆ alkoxy silane of the formula (S-I) with water (reaction scheme using the example of 3-aminopropyltriethoxysilane):

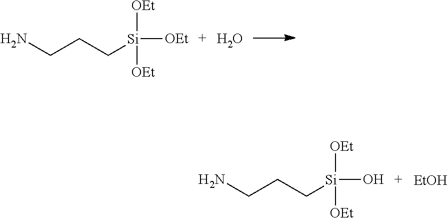

depending on the amount of water used, the hydrolysis reaction can also take place several times per C₁-C₆ alkoxy silane used

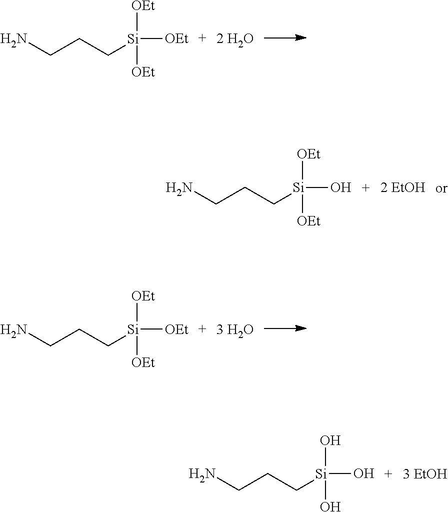

hydrolysis of C₁-C₆ alkoxy silane of the formula (S-IV) with water (reaction scheme using the example of methyltrimethoxysilane):

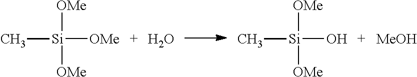

depending on the amount of water used, the hydrolysis reaction can also take place several times per C₁-C₆ alkoxy silane used

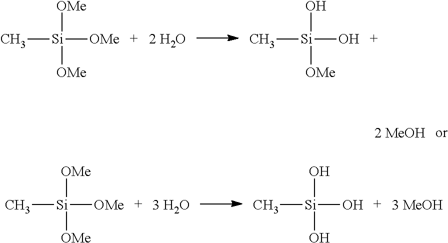

Possible condensation reactions are for example (shown by the mixture of (3-aminopropyl)triethoxysilane and methyltrimethoxysilane):

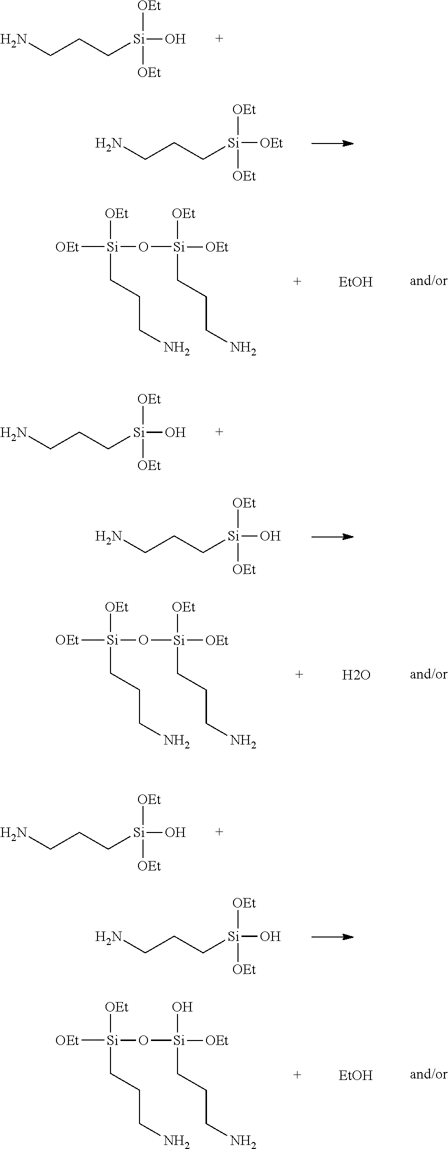

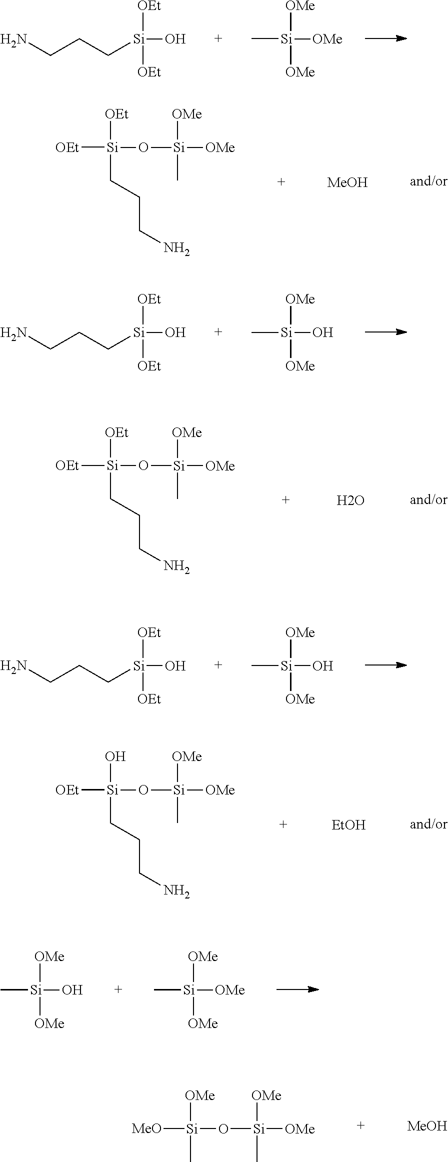

In the above exemplary reaction schemes the condensation to a dimer is shown in each case, but further condensations to oligomers with several silane atoms are also possible and also preferred.

Both partially hydrolyzed and completely hydrolyzed C₁-C₆-alkoxysilanes of the formula (S-I) can participate in these condensation reactions, which undergo condensation with partially or also completely hydrolyzed C₁-C₆-alkoxysilanes of the formula (S-I) which have not yet reacted. In this case, the C₁-C₆ alkoxysilanes of formula (S-I) react with themselves.

Furthermore, both partially hydrolyzed and completely hydrolyzed C₁-C₆-alkoxysilanes of the formula (S-I) can also participate in the condensation reactions, which undergo condensation with not yet reacted, partially or also completely hydrolyzed C₁-C₆-alkoxysilanes of the formula (S-IV). In this case, the C₁-C₆ alkoxysilanes of formula (S-I) react with the C₁-C₆ alkoxysilanes of formula (S-IV).

Furthermore, both partially hydrolyzed and completely hydrolyzed C₁-C₆-alkoxysilanes of the formula (S-IV) can also participate in the condensation reactions, which undergo condensation with not yet reacted, partially or also completely hydrolyzed C₁-C₆-alkoxysilanes of the formula (S-IV). In this case, the C₁-C₆ alkoxysilanes of formula (S-IV) react with themselves.

The composition (A) according to the present disclosure may comprise one or more organic C₁-C₆ alkoxysilanes (A2) in various proportions. This is determined by the expert depending on the desired thickness of the silane coating on the keratin material and the amount of keratin material to be treated.

Particularly storage-stable preparations with very good dyeing results in use could be obtained if the composition (A) contains—based on its total weight—one or more organic C₁-C₆-alkoxysilanes (A2) and/or the condensation products thereof in a total amount of from about 30.0 to about 85.0% by weight, preferably from about 35.0 to about 80.0% by weight, more preferably from about 40.0 to about 75.0% by weight, still more preferably from about 45.0 to about 70.0% by weight and highly preferably from about 50.0 to about 65.0% by weight.

In a further version, a highly preferred process is exemplified in that the first composition (A) comprises—based on the total weight of the composition (A)—one or more organic C₁-C₆ alkoxysilanes (A2) and/or the condensation products thereof in a total amount of from about 30.0 to about 85.0% by weight, preferably from about 35.0 to about 80.0% by weight, more preferably from about 40.0 to about 75.0% by weight, still more preferably from about 45.0 to about 70.0% by weight, and highly preferably from about 50.0 to about 65.0% by weight.

### Other Cosmetic Ingredients in the Composition (A)

In principle, the composition (A) may also comprise one or more further cosmetic ingredients.

The cosmetic ingredients which may be optionally used in the composition (A) may be any suitable ingredients to impart further beneficial properties to the product. For example, the composition (A) may contain a solvent, a thickening or film-forming polymer, a surface-active compound from the group of nonionic, cationic, anionic or zwitterionic/amphoteric surfactants, coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

The selection of these other substances will be made by the specialist according to the desired properties of the agents. With regard to other optional components and the quantities of these components used, explicit reference is made to the relevant manuals known to the specialist.

However, as described previously, the organic C₁-C₆ alkoxysilanes (A2) can react not only with water but also with other cosmetic ingredients. To avoid these undesirable reactions, the preparations (A) with alkoxy silanes therefore preferably contain no other ingredients or only the selected ingredients which have proved to be chemically inert to the C₁-C₆ alkoxy silanes. In this context, it has proved particularly preferred to use in composition (A) a cosmetic ingredient selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane.

In another particularly preferred version, a process according to the present disclosure is exemplified in that the first composition (A) comprises at least one cosmetic ingredient selected from the group of hexamethyldisiloxane, octamethyltrisiloxane, decamethyltetrasiloxane, hexamethylcyclotrisiloxane, octamethylcyclotetrasiloxane and decamethylcyclopentasiloxane.

Hexamethyldisiloxane has the CAS number 107-46-0 and can be purchased commercially from SIGMA-ALDRICH®, for example.

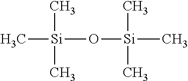

Octamethyltrisiloxane has the CAS number 107-51-7 and is also commercially available from SIGMA-ALDRICH®.

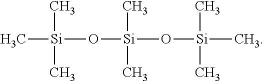

Decamethyltetrasiloxane has the CAS number 141-62-8 and is also commercially available from SIGMA-ALDRICH®.

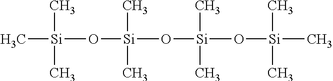

### Hexamethylcyclotrisiloxane has the CAS No 541-05-9.

### Octamethylcyclotetrasiloxane has the CAS No 556-67-2.

### Decamethylcyclopentasiloxane has the CAS No 541-02-6.

The use of hexamethyldisiloxane in composition (A) has been found to be particularly preferred. Particularly preferably, hexamethyldisiloxane is present—based on the total weight of composition (A)—in amounts of about 10.0 to about 50.0% by weight, preferably about 15.0 to about 45.0% by weight, further preferably about 20.0 to about 40.0% by weight, still further preferably about 25.0 to about 35.0% by weight and most preferably about 31.0 to about 34.0% by weight in composition (A).

In a further particularly preferred version, a method is exemplified in that the first composition (A) contains—based on the total weight of the composition (A)—about 10.0 to about 50.0% by weight, preferably about 15.0 to about 45.0% by weight, further preferably about 20.0 to about 40.0% by weight, still further preferably about 25.0 to about 35.0% by weight and highly preferably about 31.0 to about 34.0% by weight of hexamethyldisiloxane.

### Water Content (B1) in the Composition (B)

Typical of the process according to the present disclosure is the application of a second composition (B) to the keratinous material, in particular to human hair.

When applied to the keratinous material, compositions (A) and (B) come into contact, this contact being particularly preferably established by prior mixing of the two compositions (A) and (B). Mixing (A) and (B) produces the keratin treatment agent ready for use, i.e. the silane blend (A) which is stable or capable of being stored is converted into its reactive form by contact with (B). Mixing of compositions (A) and (B) starts a polymerization reaction originating from the alkoxy-silane monomers or alkoxy-silane oligomers, which finally leads to the formation of the film or coating on the keratin material.

The more water comes into contact with the organic C₁-C₆ alkoxy silane(s), the greater the extent of the polymerization reaction. For example, if the composition (B) contains a lot of water, the monomeric or oligomeric silane condensates previously present in the low-water composition (A) now polymerize very rapidly to form polymers of higher or high molecular weight. The high molecular weight silane polymers then form the film on the keratinous material. For this reason, water (B1) is an essential ingredient of the present disclosure of composition (B).

The amount of water in the composition (B) can help determine the polymerization rate of the C₁-C₆ organic alkoxy silanes (A2) at the time of application. In order to ensure an even color result when dyeing the entire head, the polymerization speed, i.e., the speed at which the coating is formed, should not be too high. For this reason, it has been found to be particularly preferable not to select too high a quantity of water in composition (B).

Particularly uniform colorations on the entire head could be obtained if the composition (B)—based on the total weight of the composition (B)—contains about 5.0 to about 90.0% by weight, preferably about 15.0 to about 85.0% by weight, more preferably about 25.0 to about 80.0% by weight, still more preferably about 35.0 to about 75.0% by weight and highly preferably about 45.0 to about 70.0% by weight of water (B1).

In another particularly preferred version, a process according to the present disclosure is exemplified in that the second composition (B) comprises—based on the total weight of the composition (B)—from about 5.0 to about 90.0% by weight, preferably from about 15.0 to about 85.0% by weight, more preferable from about 25.0 to about 80.0% by weight, still more preferable from about 35.0 to about 75.0% by weight, and highly preferable from about 45.0 to about 70.0% by weight of water (B1).

### Surfactants (B2) and (B3) in Composition (B)

The composition (B) is further exemplified by its content of at least a first surfactant (B2) and a second surfactant (B3). Both surfactants (B2) and (B3) are structurally different from each other, which means that both surfactants (B2) and (B3) are different in terms of their chemical structure.

Surprisingly, the use of the two surfactants (B2) and (B3) was found to optimize the reaction rate of the organic C₁-C₆ alkoxy-silanes such that a uniform coloration over the entire head was possible.

Due to its content of water (B1) and surfactants (B2) and (B3), composition (B) is in the form of an emulsion or a system composed of micelles. Without being committed to this theory, it is believed that the C₁-C₆ alkoxy-silanes (A2) are embedded in the micelles or the hydrophobic areas of the emulsion. In this way, the immediate environment of the C₁-C₆ alkoxy-silanes (A2) is hydrophobized. Since it is assumed that the hydrolysis and/or condensation reaction rate of the C₁-C₆ alkoxysilanes is slower in a non-polar environment, the reactivity of the C₁-C₆ alkoxysilanes can be reduced in this way and the formation of the film or coating on the keratin material slowed down.

The term surfactants (T) refers to surface-active substances that can form adsorption layers on surfaces and interfaces or aggregate in bulk phases to form micelle colloids or lyotropic mesophases. Specialists generally distinguish anionic surfactants including a hydrophobic residue and a negatively charged hydrophilic head group, amphoteric surfactants which carry both a negative and a compensating positive charge, cationic surfactants which have a positively charged hydrophilic group in addition to a hydrophobic residue, and nonionic surfactants which have a hydrophobic residue and furthermore no charges but molecular groupings with strong dipole moments which are strongly hydrated in aqueous solution.

In another particularly preferred version, a process according to the present disclosure is exemplified in that the second composition (B) comprises at least one first surfactant (B2) chosen from the group of nonionic, cationic, amphoteric or anionic surfactants.

In another particularly preferred version, a process according to the present disclosure is exemplified in that the second composition (B) comprises at least one second surfactant (B3) chosen from the group of nonionic, cationic, amphoteric or anionic surfactants.

It has been shown to be particularly preferred to use at least one nonionic surfactant from group (B2) as the surfactant.

In the context of a further highly preferred version, a process according to the present disclosure is exemplified in that the second composition (B) comprises at least one first surfactant (B2) chosen from the group of nonionic surfactants.

It has proved to be particularly preferred to use at least one nonionic surfactant from group (B3) as the surfactant.

In another particularly preferred version, a process according to the present disclosure is exemplified in that the second composition (B) comprises at least one second surfactant (B3) chosen from the group of nonionic surfactants.

The use of two structurally different nonionic surfactants (B2) and (B3) in composition (B) has been found to be particularly preferred for solving the problem according to the present disclosure.

In another particularly preferred version, a method according to the present disclosure is exemplified in that the second composition (B) comprises
at least one first nonionic surfactant (B2), and
at least one second nonionic surfactant (B3) which is structurally different from the first nonionic surfactant (B2).

Non-ionic surfactants contain, for example, a polyol group, a polyalkylene glycol ether group or a combination of polyol and polyglycol ether group as a hydrophilic group. Such links include
Addition products of about 2 to about 50 mol ethylene oxide and/or 0 to about 5 mol propylene oxide to linear and branched fatty alcohols with 6 to about 30 C atoms, the fatty alcohol polyglycol ethers or the fatty alcohol polypropylene glycol ethers or mixed fatty alcohol polyethers,
Addition products of about 2 to about 50 moles of ethylene oxide and/or 0 to about 5 moles of propylene oxide to linear and branched fatty acids having 6 to about 30 carbon atoms, the fatty acid polyglycol ethers or the fatty acid polypropylene glycol ethers or mixed fatty acid polyethers,
Addition products of about 2 to about 50 mol ethylene oxide and/or 0 to about 5 mol propylene oxide to linear and branched alkylphenols having 8 to about 15 C atoms in the alkyl group, the alkylphenol polyglycol ethers or the alkylpolypropylene glycol ethers or mixed alkylphenol polyethers,
addition products of about 2 to about 50 moles of ethylene oxide and/or 0 to about 5 moles of propylene oxide to linear and branched fatty alcohols containing 8 to about 30 carbon atoms, to fatty acids containing 8 to about 30 carbon atoms and to alkylphenols containing 8 to about 15 carbon atoms in the alkyl group, terminated by a methyl or C₂-C₆ alkyl group, such as the grades obtainable under the sales names DEHYDOL® LS, DEHYDOL® LT (COGNIS™)
C₁₂-C₃₀ fatty acid mono- and diesters of addition products of about 1 to about 30 moles of ethylene oxide to glycerol,
addition products of about 5 to about 60 mol ethylene oxide to castor oil and hardened castor oil,
polyol fatty acid esters, such as the commercial product HYDAGEN® HSP (COGNIS™) or SOVERMOL® types (COGNIS™)
alkoxylated triglycerides,
alkoxylated fatty acid alkyl esters of the formula (Tnio-1)

R₁CO—(OCH₂CHR₂)_{w}OR₃ (Tnio-1)

in which R₁CO is a linear or branched, saturated and/or unsaturated acyl radical containing 6 to about 22 carbon atoms, R₂ is hydrogen or methyl, R₃ is a linear or branched alkyl radical containing 1 to 4 carbon atoms and w is a number of 1 to about 20,
aminoxides,
hydroxy mixed ethers, as described for example in DE-OS 19738866,
sorbitan fatty acid esters and addition products of ethylene oxide to sorbitan fatty acid esters such as polysorbates,
sugar fatty acid esters and addition products of ethylene oxide to sugar fatty acid ester,
addition products of ethylene oxide to fatty acid alkanolamides and fatty amines,
Sugar surfactants of the fatty acid N-alkyl polyhydroxyalkylamide type, a nonionic surfactant of formula (Tnio-3),

R₅CO—NR₆—[Z] (Tnio-3)

in which R₅CO is an aliphatic acyl radical containing 6 to about 22 carbon atoms, R6 is hydrogen, an alkyl or hydroxyalkyl radical containing 1 to 4 carbon atoms and [Z] is a linear or branched polyhydroxyalkyl radical containing 3 to about 12 carbon atoms and 3 to about 10 hydroxyl groups. The fatty acid N-alkyl polyhydroxyalkylamides are known substances which can usually be obtained by reductive amination of a reducing sugar with ammonia, an alkylamine or an alkanolamine and subsequent acylation with a fatty acid, a fatty acid alkyl ester or a fatty acid chloride. Preferably, the fatty acid N-alkyl polyhydroxyalkylamides are derived from reducing sugars having 5 or 6 carbon atoms, in particular from glucose. The preferred fatty acid N-alkyl polyhydroxyalkylamides are therefore fatty acid N-alkylglucamides as represented by the formula (Tnio-4):

R₇CO—(NR₈)—CH₂—[CH(OH)]₄—CH₂OH (Tnio-4)

Preferably, glucamides of the formula (Tnio-4) are used as fatty acid-N-alkyl polyhydroxyalkylamides, in which R₈ represents hydrogen or an alkyl group and R₇CO represents the acyl radical of caproic acid, caprylic acid, capric acid, Lauric acid, myristic acid, palmitic acid, palmitoleic acid, stearic acid, isostearic acid, oleic acid, elaidic acid, petroselinic acid, linoleic acid, linolenic acid, arachidic acid, gadoleic acid, behenic acid or erucic acid or their technical mixtures. Particularly preferred are fatty acid N-alkylglucamides of formula (Tnio-4) obtained by reductive amination of glucose with methylamine and subsequent acylation with lauric acid or C12/14 coconut fatty acid or a corresponding derivative. Furthermore, the polyhydroxyalkylamides can also be derived from maltose and PALATINOSE™.

Other typical examples of nonionic surfactants are fatty acid amide polyglycol ethers, fatty amine polyglycol ethers, mixed ethers or mixed formals, protein hydrolysates (especially wheat-based vegetable products) and polysorbates.

By selecting very specific nonionic surfactants (B2), it was possible to obtain particularly good results with regard to the solution of the problem according to the present disclosure.

The use of at least one nonionic alkyl glycoside (B2) in composition (B) has proved to be particularly suitable.

Alkyl glycosides are generally understood to be the derivatives of monosaccharides or oligosaccharides obtained by condensation reaction of the anomeric hydroxy groups of a monosaccharide (or an anomeric hydroxy group of the oligosaccharide) with alcoholic hydroxy groups.

For example, to obtain surface active alkyl glycosides, monosaccharides can be reacted with a C₁₂-C₃₀ fatty alcohol. The surfactants of this type are called alkyl monoglycosides. Oligosaccharides can also be reacted with a corresponding C₁₂-C₃₀ fatty alcohol and the surfactants in this group are known as alkyl oligoglycosides. Alkyl monoglycosides and alkyl oligoglycosides are also grouped under the term sugar surfactants.

The use of at least one nonionic sugar surfactant (B2) of the formula (T-I) has proved to be particularly suitable,

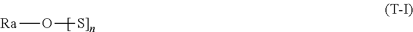

wherein
Ra represents a saturated or unsaturated, unbranched or branched C₁₂-C₃₀ alkyl group, and
n represents an integer from 1 to about 10, preferably an integer from 1 to 5, more preferable an integer from 1 to 3, and most preferable the number 1, and
S represents a sugar residue with 5 or 6 carbon atoms.

In another particularly preferred version, a process according to the present disclosure is exemplified in that the second composition (B) comprises at least one first nonionic surfactant (B2) of the formula (T-I),

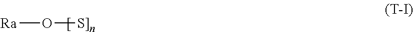

wherein
Ra represents a saturated or unsaturated, unbranched or branched C₁₂-C₃₀ alkyl group, and
n represents an integer from 1 to about 10, preferably an integer from 1 to 5, more preferable an integer from 1 to 3, and most preferable the number 1, and
S represents a sugar residue with 5 or 6 carbon atoms.

The alkyl monoglycosides or alkyl oligoglycosides can be derived from sugars from the group of aldoses and ketoses with 5 or 6 carbon atoms, preferably derived from xylose, ribose, arabinose, lyxose, allose, altrose, glucose, mannose, gulose, idose, galactose and/or talose.

The index number n in the general formula (T-I) indicates the degree of oligomerization, i.e., the distribution of mono- and oligoglycosides and stands for a number between 1 and about 10.

In the case of an alkyl monoglycoside, a monosaccharide is glycosidically linked to one of the fatty alcohols described above. In this case n stands for the number 1. The use of an alkyl monoglycoside as the first nonionic surfactant (B2) is particularly preferred.

In the case of an oligoglycoside, an oligomer formed from sugars (i.e., a di- or oligosaccharide) is glycosidically linked to one of the fatty alcohols described above. In this case, n represents a higher number such as 2, 3, 4, 5, 6, 7, 8, 9, or 10.

The radical Ra represents a saturated or unsaturated, unbranched or branched C₁₂-C₃₀ alkyl group.

Very preferably, Ra represents a saturated, unbranched or branched C₁₂-C₃₀ alkyl group, highly preferable a branched C₁₂-C₂₂ alkyl group.

In another particularly preferred version, a process according to the present disclosure is exemplified in that the second composition (B) comprises at least one first nonionic surfactant (B2) of the formula (T-I) whereby

Ra represents a saturated, branched C₁₂-C₃₀ alkyl group, highly preferable a saturated, branched C₁₂-C₂₂ alkyl group,
n represents for the number 1 and
S represents for a xylitol residue.

A sugar surfactant based on a xylitol residue represents, for example, an alkyl monoxylide or alkyl oligoxylide in which the xylitol radical is condensed via its anomeric C atom with the C₁₂-C₃₀ fatty alcohol to form the glycosidic bond:
Alkyl monoglycoside starting from beta-D-xylopyranose

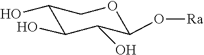

Alkyl monoglycoside starting from alpha-D-xylopyranose

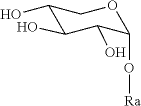

Alkylmonoglycoside starting from beta-D-xylofuranose

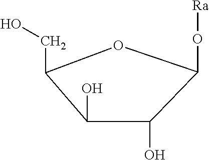

Alkylmonoglycoside starting from alpha-D-xylofuranose

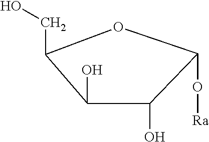

The definition of sugar radicals starting from ribose, arabinose, lyxose, allose, altrose, glucose, mannose, gulose, idose, galactose and/or talose applies analogously.

As an explicitly highly preferred first nonionic surfactant (B2), 2-octyldodecyl xyloside may be used, for example. 2-Octyldodecyl xyloside is a glycoside obtained by condensation of xylitol and 2-octyldodecanol.

By selecting the appropriate quantities of non-ionic surfactants (B2), the rate of film formation originating from the C₁-C₆ alkoxy silanes can be especially strongly co-determined. For this reason, it has been found to be particularly preferable to use one or more nonionic surfactants (B2) in very specific ranges of amounts.

It is particularly preferred if the second composition (B) comprises—based on the total weight of the composition (B)—one or more surfactants (B2) in a total amount of from about 0.5 to about 20.0% by weight, preferably from about 1.0 to about 10.0% by weight, more preferable from about 1.5 to about 8.0% by weight and most preferable from about 2.0 to about 7.0% by weight.

In the context of a further particularly preferred version, a process according to the present disclosure is exemplified in that the second composition (B) comprises—based on the total weight of the composition (B)—one or more surfactants (B2) in a total amount of from about 0.5 to about 20.0% by weight, preferably from about 1.0 to about 10.0% by weight, more preferably from about 1.5 to about 8.0% by weight, and most preferably from about 2.0 to about 7.0% by weight.

Another feature of the compositions (B) used in the process according to the present disclosure is that, in addition to the first surfactant or surfactants of group (B2), they additionally contain at least one more surfactant (B3), the surfactant or surfactants of group (B3) being structurally different from the surfactant or surfactants of group (B2).

The second surfactant or the second surfactant group (B3) is also highly preferred to be a non-ionic surfactant. Suitable nonionic surfactants have been described previously.

Explicitly excellent results were obtained when a second composition (B) according to the present disclosure was used which contained at least a second nonionic surfactant (B3) of formula (T-II),

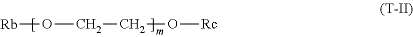

wherein
Rb, Rc independently of one another represent a saturated, unbranched or branched, unsubstituted or substituted, C₁₂-C₃₀-alkanoyl group,
m represents an integer from 2 to about 60, preferably an integer from about 10 to about 50,

more preferably an integer from about 15 to about 40 and highly preferably an integer from about 25 to about 35.

In another highly preferred version, a process according to the present disclosure is exemplified in that the second composition (B) comprises at least one second nonionic surfactant (B3) of formula (T-II),

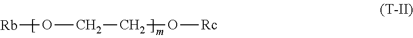

wherein
Rb, Rc independently of one another represent a saturated, unbranched or branched, unsubstituted or substituted, C₁₂-C₃₀-alkanoyl group,
m represents an integer from 1 to about 60, preferably an integer from about 10 to about 50, more preferably an integer from about 15 to about 40 and highly preferably an integer from about 25 to about 35.

In the surfactants (B3) of the formula (T-II), the radicals Rb, Rc independently of one another represent a saturated, unbranched or branched, unsubstituted or substituted, C₁₂-C₃₀-alkanoyl group. The C₁₂-C₃₀ alkanoyl group may alternatively be referred to as the C₁₂-C₃₀ acyl group.

In the case where the index number m represents 1, the compound of the formula (T-II) is 1,2-ethanediol in which both hydroxyl groups are esterified with C₁₂-C₃₀ fatty acids, where these fatty acids may be saturated, unbranched or branched, unsubstituted or substituted as defined for Rb and Rc. If the C₁₂-C₃₀ alkanoyl group(s) are substituted, it is highly preferable if they carry one or more hydroxy groups as substituents.

For example, Rb and Rc may independently represent one of the following structures:

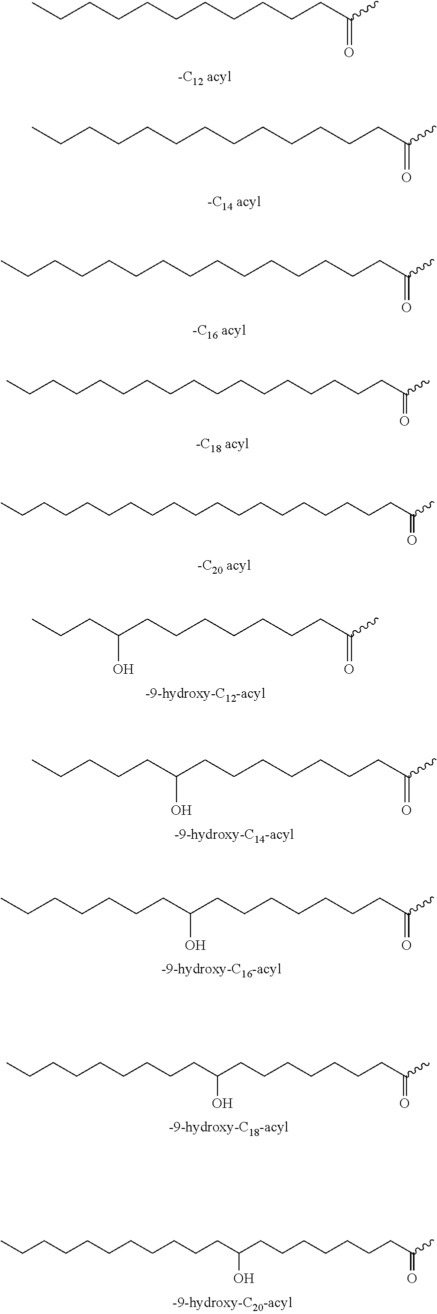

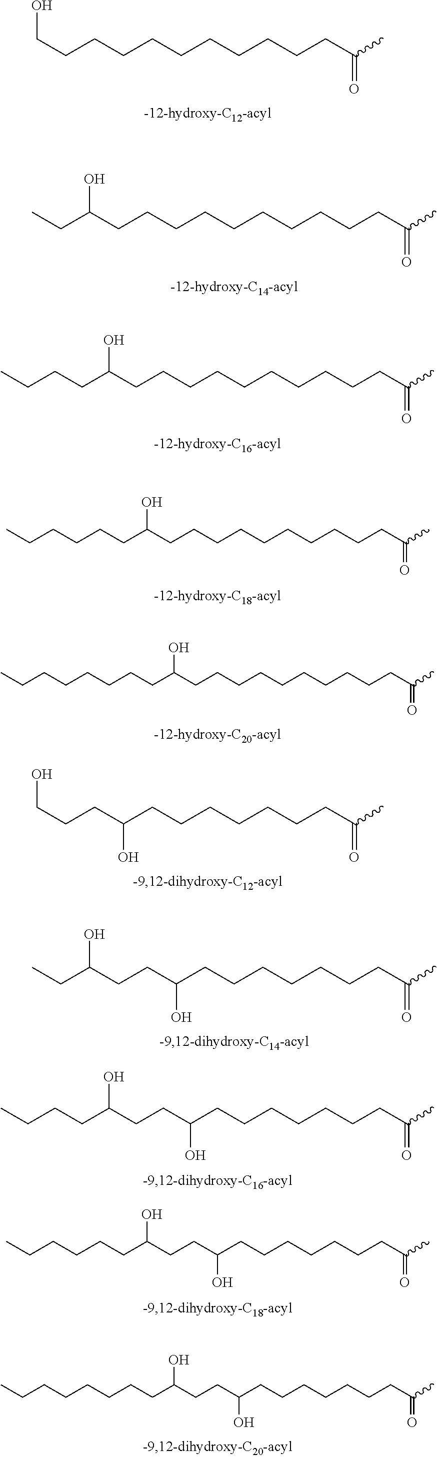

One particularly suitable surfactant of structure (T-II) is the substance polyoxyethylene (30) dipolyhydroxystearate. In this case, Rb and Rc both represent a 12-hydroxy-C₁₆-acyl group and the index number m represents 30.

By selecting the appropriate quantities of non-ionic surfactants (B3), the rate of film formation originating from the C₁-C₆ alkoxy silanes can be especially strongly co-determined. For this reason, it has been found to be particularly preferable to use one or more nonionic surfactants (B3) in very specific ranges of amounts.

It is particularly preferred if the second composition (B) comprises—based on the total weight of the composition (B)—one or more surfactants (B3) in a total amount of from about 0.5 to about 20.0% by weight, preferably from about 1.0 to about 10.0% by weight, more preferable from about 1.5 to about 8.0% by weight and most preferable from about 2.0 to about 7.0% by weight.

In the context of a further particularly preferred version, a process according to the present disclosure is exemplified in that the second composition (B) comprises—based on the total weight of the composition (B)—one or more surfactants (B3) in a total amount of from about 0.5 to about 20.0% by weight, preferably from about 1.0 to about 10.0% by weight, more preferably from about 1.5 to about 8.0% by weight, and most preferably from about 2.0 to about 7.0% by weight.

### Fat Components in the Composition (B)

In addition to surfactants (B2) and (B3), composition (B) may optionally comprise one or more further hydrophobic components or fatty components.

The fatty components are also hydrophobic substances which can form emulsions in the presence of water with the formation of micelle systems. In analogy to the terpenes, it is also assumed in this context that the C₁-C₆ alkoxysilanes—either in the form of their monomers or optionally in the form of their condensed oligomers—are embedded in this hydrophobic environment or in the micelle systems, so that the polarity of their environment changes. Due to the hydrophobic nature of the fatty components, the environment of the C₁-C₆ alkoxysilanes is also hydrophobized. It is assumed that the polymerization reaction of the C₁-C₆ alkoxy silanes leading to the film or coating takes place in an environment of reduced polarity at a reduced rate.

Particularly preferred, the fatty ingredients present in the composition (B) are selected from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

In a highly preferred version, a process according to the present disclosure is exemplified in that the second composition (B) comprises one or more fat constituents from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

In this context, highly preferred fat constituents are understood to be constituents from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons. For the purposes of the present disclosure, only non-ionic substances are explicitly considered as fat components. Charged compounds such as fatty acids and their salts are not considered as fat constituents.

The C₁₂-C₃₀ fatty alcohols may be saturated, mono- or polyunsaturated, linear or branched fatty alcohols with 12 to 30 C atoms.

Examples of preferred linear, saturated C₁₂-C₃₀ fatty alcohols include dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol), and/or behenyl alcohol (docosan-1-ol).

Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol).

The preferred representatives for branched fatty alcohols are 2-octyl-dodecanol, 2-hexyl-dodecanol and/or 2-butyl-dodecanol.

2-octyl-dodecanol is explicitly highly preferred.

By selecting particularly well-suited fatty components, the polarity of the composition (B) can be optimally adjusted and the polymerization rate of the C₁-C₆ alkoxysilanes can be particularly well adapted to the respectively selected application conditions.

In this context, it has been found that in particular the use of at least one C₁₂-C₃₀ fatty alcohol in composition (B) creates an emulsion system in which the alkoxysilanes (A2) can be especially well embedded.

In one version, extremely good results were obtained when the second composition (B) comprises one or more C₁₂-C₃₀ fatty alcohols selected from the group of dodecan-1-ol (dodecyl alcohol, lauryl alcohol), Tetradecan-1-ol (tetradecyl alcohol, myristyl alcohol), Hexadecan-1-ol (hexadecyl alcohol, cetyl alcohol, palmityl alcohol), Octadecan-1-ol (Octadecyl alcohol, Stearyl alcohol), Arachyl alcohol (Eicosan-1-ol), Heneicosyl alcohol (Heneicosan-1-ol), Behenyl alcohol (docosan-1-ol), (9Z)-Octadec-9-en-1-ol (oleyl alcohol), (9E)-Octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), Gadoleyl alcohol ((9Z)-Eicos-9-en-1-ol), Arachidone alcohol ((5Z,8Z,11Z,14Z)-Eicosa-5,8,11,14-tetraen-1-ol), Erucyl alcohol ((13Z)-Docos-13-en-1-ol), brassidyl alcohol ((13E)-docosen-1-ol) 2-octyl-dodecanol, 2-hexyl-dodecanol and/or 2-butyl-dodecanol.

In an extremely preferred version, a process according to the present disclosure is exemplified in that the second composition (B) comprises one or more C₁₂-C₃₀ fatty alcohols selected from the group of

dodecan-1-ol (dodecyl alcohol, lauryl alcohol),
tetradecan-1-ol (tetradecyl alcohol, myristyl alcohol),
hexadecan-1-ol (hexadecyl alcohol, cetyl alcohol, palmityl alcohol),
octadecan-1-ol (octadecyl alcohol, stearyl alcohol),
arachyl alcohol (eicosan-1-ol),
heneicosyl alcohol (heneicosan-1-ol),
behenyl alcohol (docosan-1-ol),
(9Z)-Octadec-9-en-1-ol (oleyl alcohol),
(9E)-Octadec-9-en-1-ol (elaidyl alcohol),
(9Z,12Z)-octadeca-9,12-dien-1-ol (linoleyl alcohol),
(9Z,12Z,15Z)-octadeca-9,12,15-trien-1-ol (linolenoyl alcohol),
Gadoleyl alcohol ((9Z)-Eicos-9-en-1-ol),
Arachidonic alcohol ((5Z,8Z,11Z,14Z)-Eicosa-5,8,11,14-tetraen-1-ol),
Erucyl alcohol ((13Z)-docos-13-en-1-ol),
Brassidyl alcohol ((13E)-docosen-1-ol),
2-octyl-dodecanol,
2-hexyl dodecanol and/or
2-butyl-dodecanol.

By selecting the appropriate amounts of C₁₂-C₃₀ fatty alcohols, the rate of film formation from the C₁-C₆ alkoxy silanes can be strongly influenced. For this reason, it has been found to be highly preferable to use one or more C₁₂-C₃₀ fatty alcohols in very specific ranges of amounts.

It is particularly preferred if the second composition (B) comprises—based on the total weight of the composition (B)—one or more C₁₂-C₃₀ fatty alcohols (B) in a total amount of from about 2.0 to about 50.0% by weight, preferably from about 4.0 to about 40.0% by weight, more preferably from about 6.0 to about 30.0% by weight, still more preferably from about 8.0 to about 20.0% by weight, and most preferably from about 10.0 to about 15.0% by weight.

In another particularly preferred version, a process according to the present disclosure is exemplified in that the second composition (B) comprises—based on the total weight of the composition (B)—one or more C₁₂-C₃₀ fatty alcohols (B) in a total amount of from about 2.0 to about 50.0% by weight, preferably from about 4.0 to about 40.0% by weight, more preferably from about 6.0 to about 30.0% by weight, still more preferably from about 8.0 to about 20.0% by weight, and most preferably from about 10.0 to about 15.0% by weight.

Furthermore, as a highly preferred fat ingredient, composition (B) may also comprise at least one C₁₂-C₃₀ fatty acid triglyceride which is C₁₂-C₃₀ fatty acid monoglyceride and/or C₁₂-C₃₀ fatty acid diglyceride. For the purposes of the present disclosure, a C₁₂-C₃₀ fatty acid triglyceride is understood to be the triester of the trivalent alcohol glycerol with three equivalents of fatty acid. Both structurally identical and different fatty acids within a triglyceride molecule can be involved in ester formation.

According to the present disclosure, fatty acids are understood to be saturated or unsaturated, unbranched or branched, unsubstituted or substituted C₁₂-C₃₀ carboxylic acids. Unsaturated fatty acids can be monounsaturated or polyunsaturated. In the case of an unsaturated fatty acid, its C—C double bond(s) may have the cis or trans configuration.

Fatty acid triglycerides are particularly suitable in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

The fatty acid triglycerides may also be of natural origin. The fatty acid triglycerides present in soybean oil, peanut oil, olive oil, sunflower oil, macadamia nut oil, moringa oil, apricot kernel oil, marula oil and/or optionally hydrogenated castor oil, or mixtures thereof, are particularly suitable for use in the product according to the present disclosure.

A C₁₂-C₃₀ fatty acid monoglyceride is the monoester of the trihydric alcohol glycerol with one equivalent of fatty acid. In this case, either the central hydroxy group of the glycerol or the terminal hydroxy group of the glycerol may be esterified with the fatty acid.

C₁₂-C₃₀ fatty acid monoglycerides are particularly suitable in which a hydroxyl group of glycerol is esterified with a fatty acid, the fatty acids being selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], or nervonic acid [(15Z)-tetracos-15-enoic acid].

A C₁₂-C₃₀ fatty acid diglyceride is the diester of the trivalent alcohol glycerol with two equivalents of fatty acid. Here, either the middle and one terminal hydroxy group of glycerol may be esterified with two equivalents of fatty acid, or both terminal hydroxy groups of glycerol may be esterified with one fatty acid each. The glycerol can be esterified with two structurally identical or two different fatty acids.

Fatty acid diglycerides are particularly suitable in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

Particularly good results were obtained when composition (B) contained at least one C₁₂-C₃₀ fatty acid monoglyceride selected from the monoesters of glycerol with one equivalent of fatty acid selected from the group of dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), Petroselic acid [(Z)-6-octadecenoic acid], Palmitoleic acid [(9Z)-Hexadec-9-enoic acid], Oleic acid [(9Z)-Octadec-9-enoic acid], Elaidic acid [(9E)-Octadec-9-enoic acid], Erucic acid [(13Z)-Docos-13-enoic acid], Linoleic acid [(9Z, 12Z)-Octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-icosa-5,8,11,14-tetraenoic acid] and/or nervonic acid [(15Z)-tetracos-15-enoic acid].

In a particularly preferred version, a process according to the present disclosure is exemplified in that the second composition (B) comprises at least one C₁₂-C₃₀ fatty acid monoglyceride selected from the monoesters of glycerol with one equivalent of fatty acid selected from the group of dodecanoic acid, tetradecanoic acid, hexadecanoic acid, tetracosanoic acid, octadecanoic acid, eicosanoic acid and/or docosanoic acid.

The choice of suitable amounts of C₁₂-C₃₀ fatty acid mono-, C₁₂-C₃₀ fatty acid di- and/or C₁₂-C₃₀ fatty acid triglycerides can also have a particularly strong influence on the rate of film formation from the C₁-C₆ alkoxy silanes. For this reason, it has been found to be particularly preferred to use one or more C₁₂-C₃₀ fatty acid mono-, C₁₂-C₃₀ fatty acid di- and/or C₁₂-C₃₀ fatty acid triglycerides in very specific amount ranges in composition (B).

With regard to the solution of the problem according to the present disclosure, it proved to be highly preferable if the second composition (B) contained—based on the total weight of the composition (B)—one or more C₁₂-C₃₀ fatty acid mono-, C₁₂-C₃₀ fatty acid di- and/or C₁₂-C₃₀ fatty acid triglycerides in a total amount of from about 0.1 to about 20.0% by weight, preferably from about 0.3 to about 15.0% by weight, more preferably from about 0.5 to about 10.0% by weight and highly preferably from about 0.8 to about 5.0% by weight.

In a highly preferred version, a process according to the present disclosure is exemplified in that the second composition (B) comprises—based on the total weight of the composition (B)—one or more C₁₂-C₃₀ fatty acid mono-, C₁₂-C₃₀ fatty acid di- and/or C₁₂-C₃₀ fatty acid triglycerides in a total amount of from about 0.1 to about 20.0% by weight, preferably from about 0.3 to about 15.0% by weight, more preferably from about 0.5 to about 10.0% by weight and highly preferably from about 0.8 to about 5.0% by weight.

The C₁₂-C₃₀ fatty acid mono-, C₁₂-C₃₀ fatty acid di- and/or C₁₂-C₃₀ fatty acid triglycerides may be used as sole fat components in the compositions (B). However, it is particularly C₁₂-C₃₀ preferred to incorporate at least one C₁₂-C₃₀ fatty acid mono-, C₁₂-C₃₀ fatty acid di- and/or C₁₂-C₃₀ fatty acid triglyceride in combination with at least one C₁₂-C₃₀ fatty alcohol into composition (B).

Furthermore, as a highly preferred fatty ingredient, the composition (B) may also comprise at least one hydrocarbon.

Hydrocarbons are compounds exclusively formed of the atoms carbon and hydrogen with 8 to about 80 C-atoms. In this context, aliphatic hydrocarbons such as mineral oils, liquid paraffin oils (e.g. paraffinum liquidum or paraffinum perliquidum), isoparaffin oils, semisolid paraffin oils, paraffin waxes, hard paraffin (paraffinum solidum), petrolatum and polydecenes are particularly preferred.

Liquid paraffin oils (paraffinum liquidum and paraffinum perliquidum) have proved to be particularly suitable in this context. Especially preferred, the hydrocarbon is paraffinum liquidum, also known as white oil. Paraffinum Liquidum is a mixture of purified, saturated, aliphatic hydrocarbons, including mainly hydrocarbon chains with a C-chain distribution of about 25 to about 35 C-atoms.

Particularly good results were obtained when composition (B) contained at least one hydrocarbon selected from the group of mineral oils, liquid paraffin oils, isoparaffin oils, semisolid paraffin oils, paraffin waxes, hard paraffin (paraffinum solidum), petrolatum and polydecenes.

In a highly preferred version, a process according to the present disclosure is exemplified in that the second composition (B) comprises at least one fatty constituent selected from the group of hydrocarbons.

The speed of film formation from the C₁-C₆ alkoxy silanes can also be particularly strongly influenced by the choice of suitable quantities of hydrocarbons. For this reason, it has been shown to be particularly preferred to use one or more hydrocarbons) in very specific ranges of amounts in the composition (B).

With regard to the solution of the problem according to the present disclosure, it has proved to be particularly preferable if the second composition (B)—based on the total weight of the composition (B)—contained one or more hydrocarbons in a total amount of from about 0.5 to about 20.0% by weight, preferably from about 1.0 to about 15.0% by weight, more preferably from about 1.5 to about 10.0% by weight and extremely preferably from about 2.0 to about 8.0% by weight.

In a particularly preferred version, a process according to the present disclosure is exemplified in that the second composition (B) comprises—based on the total weight of the composition (B)—one or more hydrocarbons in a total amount of from about 0.5 to about 20.0% by weight, preferably from about 1.0 to about 15.0% by weight, more preferably from about 1.5 to about 10.0% by weight and highly preferable from about 2.0 to about 8.0% by weight.

The hydrocarbon(s) may be used as the sole fatty ingredients in compositions (B). However, it is particularly preferred to incorporate at least one hydrocarbon in combination with at least one other constituent in the compositions (B).

It is particularly preferred if the composition (B) comprises at least one fatty constituent from the group of C₁₂-C₃₀ fatty alcohols and at least one other fatty constituent from the group of hydrocarbons.

### Solvent in the Composition (B)

Further work leading to the present disclosure has shown that the use of at least one protic solvent in composition (B) also reduces the rate of reaction of the C₁-C₆ alkoxy silanes when in contact with composition (A).

Protic solvents have at least one hydroxy group. Without being committed to this theory, it is assumed that the solvents can also react with the C₁-C₆ alkoxysilanes via their hydroxyl group(s), but that the reaction between solvents and C₁-C₆ alkoxysilanes proceeds more slowly than the analogous reaction between water and C₁-C₆ alkoxysilanes. In summary, the hydrolysis and/or condensation reaction of the C₁-C₆ alkoxy silanes is reduced in this way.

For example, well-suited solvents may include 1,2-propylene glycol, 1,3-propylene glycol, ethylene glycol, 1,2-butylene glycol, dipropylene glycol, ethanol, isopropanol, diethylene glycol monoethyl ether, glycerol, phenoxyethanol and/or benzyl alcohol.

In another particularly preferred version, a process according to the present disclosure is exemplified in that the second composition (B) comprises at least one solvent selected from the group of 1,2-propylene glycol, 1,3-propylene glycol, ethylene glycol, 1,2-butylene glycol, dipropylene glycol, ethanol, isopropanol, diethylene glycol monoethyl ether, glycerol, phenoxyethanol and/or benzyl alcohol.

Compositions (B) containing 1,2-propylene glycol as solvent are particularly preferred.

1,2-Propylene glycol is alternatively known as 1,2-propanediol and has the CAS numbers 57-55-6 [(RS)-1,2-dihydroxypropane], 4254-14-2 [(R)-1,2-dihydroxypropane] and 4254-15-3 [(S)-1,2-dihydroxypropane]. Ethylene glycol is alternatively known as 1,2-ethanediol and has the CAS number 107-21-1. Glycerol is alternatively known as 1,2,3-propanetriol and has the CAS number 56-81-5. Phenoxyethanol has the Cas number 122-99-6.

All of the solvents described above are commercially available from various chemical suppliers such as ALDRICH® or FLUKA®.

By using the aforementioned solvents in suitable application quantities, the rate of film formation originating from the C₁-C₆ alkoxy silanes are particularly strongly co-determined. For this reason, it has proved particularly preferable to use one or more solvents in very specific quantity ranges.

It is particularly preferred if the second composition (B) comprises—based on the total weight of the composition (B)—one or more solvents in a total amount of from about 1.0 to about 35.0% by weight, preferably from about 4.0 to about 25.0% by weight, more preferably from about 8.0 to about 20.0% by weight, and most preferably from about 10.0 to about 15.0% by weight.

It is particularly preferred if the second composition (B) contains—based on the total weight of the composition (B)—one or more solvents from the group of 1,2-propylene glycol, 1,3-propylene glycol, ethylene glycol, 1,2-butylene glycol, dipropylene glycol, ethanol, isopropanol, diethylene glycol monoethyl ether, glycerol, phenoxyethanol and/or benzyl alcohol in a total amount of from about 1.0 to about 35.0% by weight, preferably from about 4.0 to about 25.0% by weight, more preferably from about 8.0 to about 20.0% by weight, and most preferably from about 10.0 to about 15.0% by weight.

### Other Cosmetic Ingredients in the Composition (B)

In addition to the highly preferred ingredients already described above, the composition (B) may further comprise one or more additional cosmetic ingredients.

The cosmetic ingredients which may be optionally used in the composition (B) may be any suitable ingredients to impart further beneficial properties to the product. For example, the composition (A) may contain a solvent, a thickening or film-forming polymer, a surface-active compound from the group of nonionic, cationic, anionic or zwitterionic/amphoteric surfactants, coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

If the process according to the present disclosure is a process for coloring keratinous material, the composition (B) may highly preferable comprise at least one coloring compound selected from the group of pigments and/or direct dyes.

The selection of these other substances will be made by the specialist according to the desired properties of the agents. With regard to other optional components and the quantities of these components used, explicit reference is made to the relevant manuals known to the specialist.

### pH Values of the Compositions in the Process

In further experiments, it has been found that the pH values of compositions (A) and/or (B) can have an influence on the hydrolysis or condensation reactions described above which take place during use. It was found that alkaline pH values in particular stop condensation at the oligomer stage. The more acidic the reaction mixture, the stronger the condensation seems to proceed and the higher the molecular weight of the silane condensates formed during condensation. For this reason, it is preferred that compositions (A) and/or (B) have a pH of from about 7.0 to about 12.0, preferably from about 7.5 to about 11.5, more preferably from about 8.5 to about 11.0, and most preferably from about 9.0 to about 11.0.

The water content of composition (A) is at most about 10.0% by weight and is preferably set even lower. In some versions, the water content of the composition (B) may also be selected to be low. Especially in the case of compositions with a very low water content, the measurement of the pH value with the usual methods known from the prior art (pH value measurement by employing glass electrodes via combination electrodes or via pH indicator paper) can prove to be difficult. For this reason, the pH values according to the present disclosure are those obtained after mixing or diluting the preparation in a weight ratio of about 1:1 with distilled water.

Accordingly, the corresponding pH is measured after, for example, 50 g of the composition according to the present disclosure has been mixed with 50 g of distilled water.

In another particularly preferred version, a process according to the present disclosure, exemplified in that the composition (A) and/or (B), after dilution with distilled water in a weight ratio of about 1:1, has a pH of from about 7.0 to about 11.5, more preferably from about 8.5 to about 11.0 and most preferably from about 9.0 to about 11.0.

To adjust this alkaline pH, it may be necessary to add an alkalizing agent and/or acidifying agent to the reaction mixture. The pH values for the purposes of the present disclosure are pH values measured at a temperature of 22° C.

For example, ammonia, alkanolamines and/or basic amino acids can be used as alkalizing agents.

Alkanolamines may be selected from primary amines having a C₂-C₆ alkyl backbone bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol.

For the purposes of the present disclosure, an amino acid is an organic compound containing in its structure at least one protonatable amino group and at least one —COOH or one —SO₃H group. Preferred amino acids are aminocarboxylic acids, especially α-(alpha)-aminocarboxylic acids and ω-aminocarboxylic acids, whereby α-aminocarboxylic acids are particularly preferred.

According to the present disclosure, basic amino acids are those amino acids which have an isoelectric point pI of greater than 7.0.

Basic α-aminocarboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred version, an agent according to the present disclosure is therefore exemplified in that the alkalizing agent is a basic amino acid from the group arginine, lysine, ornithine and/or histidine.

In addition, inorganic alkalizing agents can also be used. Inorganic alkalizing agents usable according to the present disclosure are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Highly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

In addition to the alkalizing agents described above, the specialist is familiar with common acidifying agents for fine adjustment of the pH value. According to the present disclosure, preferred acidifiers are pleasure acids, such as citric acid, acetic acid, malic acid or tartaric acid, as well as diluted mineral acids.

### Use of Compositions (A) and (B)

The method according to the present disclosure comprises applying both compositions (A) and (B) to the keratinous material. It is essential to the process that compositions (A) and (B) come into contact with each other on the keratinous material. As previously described, this contact can be made either by mixing (A) and (B) beforehand or by successively applying (A) and (B) to the keratin material.

The work leading to the present disclosure has shown that composition (B) containing water (B1) and surfactants (B2) and (B3) can have an optimum effect on the low-water silane blend (i.e., composition (A)), in particular when compositions (A) and (B) have been mixed together before use.

This mixing can be done, for example, by stirring or shaking. It is particularly advantageous to prepare the two compositions (A) and (B) separately in two containers and then, before use, to transfer the entire quantity of composition (A) from its container into the container containing the second composition (B).

In a highly preferred version, a process according to the present disclosure is exemplified in that a composition is applied to the keratinous material which has been prepared immediately before application by mixing the first composition (A) and the second composition (B).

The two compositions (A) and (B) may be mixed together in different proportions.

Especially preferred, composition (A) is used in the form of a relatively highly concentrated, low-water silane blend, which is quasi-diluted by mixing with composition (B). For this reason, it is particularly preferred to mix composition (A) with an excess by weight of composition (B). For example, 1 part by weight of (A) may be mixed with about 20 parts by weight of (B), or 1 part by weight of (A) may be mixed with about 10 parts by weight of (B), or 1 part by weight of (A) may be mixed with about 5 parts by weight of (B).

In a highly preferred version, a process according to the present disclosure is exemplified in that a composition is applied to the keratinous material which has been prepared immediately before application by mixing the first composition (A) and the second composition (B) in a quantitative ratio (A)/(B) of from about 1:5 to about 1:20.

In principle, however, it is also possible to use composition (A) in excess by weight in relation to composition (B). For example, about 20 parts by weight of (A) may be mixed with 1 part by weight of (B), or about 10 parts by weight of (A) may be mixed with 1 part by weight of (B), or about 5 parts by weight of (A) may be mixed with 1 part by weight of (B).

Furthermore, it is also conceivable to apply the compositions (A) and (B) successively to the keratinous material, so that the contact of (A) and (B) only occurs on the keratinous material. In the context of this version, preferably no washing of the keratin matrix is carried out between the application of compositions (A) and (B), i.e., no treatment of the keratin matrix with water or water and surfactants.

In one version, only both compositions (A) and (B) may be used on the keratinous material. In particular, when using the method according to the present disclosure for dyeing keratinous material, it may also be particularly preferred if not only the two compositions (A) and (B), but furthermore at least one third composition (C) is applied to the keratinous material.

In a process for coloring keratinous material, the third composition (C) may, for example, be a composition comprising at least one coloring compound selected from the group of pigments and/or direct dyes.

In the context of a further version, highly preferred is a process according to the present disclosure in which the following is applied to the keratinous material
a third composition (C) comprising

at least one coloring compound selected from the group of pigments and/or direct dyes.

Using the three compositions (A), (B) and (C), various versions are according to the present disclosure.

In one version, it is particularly preferred to prepare a mixture of the three compositions (A), (B) and (C) prior to application and then to apply this mixture to the keratin material.

In a particularly preferred version, a process according to the present disclosure is exemplified in that a composition obtained immediately before use by mixing the first composition (A) with the second composition (B) and a third composition (C) is applied to the keratinous material, the third composition (C) comprising at least one coloring compound chosen from the group of pigments and/or direct dyes.

When coloring the keratinous material, it may also be particularly preferred to prepare a mixture immediately before use by mixing the first composition (A) and the second composition (B) and to apply this mixture of (A) and (B) to the keratinous material. The third composition (C) containing the coloring compounds can then be added to the keratin material.

Within the framework of a highly preferred version, a process according to the present disclosure is exemplified in that a composition is applied to the keratinous material, which was obtained immediately before the application by mixing the first composition (A) with the second composition (B), and subsequently the composition (C) is applied to the keratinous material.

In other words, a particularly preferred process according to the present disclosure is exemplified in that, in a first step, a composition is applied to the keratinous material, which was prepared immediately before application by mixing the first composition (A) and the second composition (B), and, in a second step, the third composition (C) is applied to the keratinous material.

In addition to compositions (A) and (B)—or (A), (B) and (C)—a fourth composition (D) can also be applied to the keratin material as part of the process according to the present disclosure. The application of the fourth composition (D) is particularly preferred in a dyeing process in order to reseal the previously obtained colorations. For this sealing, the composition (D) may contain, for example, at least one film-forming polymer.

In other words, further a highly preferred process according to the present disclosure is one in which the following is applied to the keratinous material
a fourth composition (D) comprising

at least one film-forming polymer.

### Coloring Compounds

When compositions (A) and (B)—or additionally optionally (C) and/or (D)—are used in a dyeing process, one or more coloring compounds may be employed.

In particular, the preparation (B) and/or the optional preparation (C) may additionally comprise at least one color-imparting compound.

The colorant compound or compounds may preferably be selected from pigments, direct dyes, oxidation dyes, photochromic dyes and thermochromic dyes, more preferably pigments and/or direct dyes.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be visually assessed due to the high intensity of the pigment, which may be finely dispersed, the mixture is filtered. If a proportion of undissolved pigments remains on the filter paper, the solubility of the pigment is below 0.5 g/L.

Suitable color pigments can be of inorganic and/or organic origin.

In a preferred version, an agent according to the present disclosure is exemplified in that it contains at least one coloring compound from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. In particular, preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Coloring compounds from the group of pigments which are also particularly preferred according to the present disclosure are colored pearlescent pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

In a particularly preferred version, a process according to the present disclosure is exemplified in that the composition (B) and/or the composition (C) comprise at least one coloring compound chosen from the group of inorganic pigments chosen from the group of colored metal oxides, metal hydroxides, metal oxide hydrates, silicates, metal sulfides, complex metal cyanides, metal sulphates, bronze pigments and/or colored mica- or mica-based pigments coated with at least one metal oxide and/or a metal oxychloride.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred version, the composition (B) and/or the composition (C) according to the present disclosure is exemplified in that it comprises at least one coloring compound chosen from the group of pigments chosen from the group of colored metal oxides, metal hydroxides, metal oxide hydrates, silicates, metal sulfides, complex metal cyanides, metal sulphates, bronze pigments and/or from mica- or mica-based coloring compounds coated with at least one metal oxide and/or a metal oxychloride.

In a further preferred version, a composition (B) and/or composition (C) according to the present disclosure is exemplified in that it comprises at least one coloring compound selected from mica- or mica-based pigments coated with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfo silicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names RONA®, COLORONA®, XIRONA®, DICHRONA® and TIMIRON® from MERCK®, ARIABEL® and UNIPURE® from SENSIENT®, PRESTIGE® from ECKART® Cosmetic Colors and SUNSHINE® from Sunstar.

Particularly highly preferred color pigments with the trade name COLORONA® are, for example:

### COLORONA® Copper, MERCK®, MICA, CI 77491 (IRON OXIDES)

### COLORONA® Passion Orange, MERCK®, Mica, CI 77491 (Iron Oxides), Alumina

### COLORONA® Patina Silver, MERCK®, MICA, CI 77499 (IRON OXIDES), CI 77891 (TITANIUM DIOXIDE)

### COLORONA® RY, MERCK®, CI 77891 (TITANIUM DIOXIDE), MICA, CI 75470 (CARMINE)

### COLORONA® Oriental Beige, MERCK®, MICA, CI 77891 (TITANIUM DIOXIDE), CI 77491 (IRON OXIDES)

### COLORONA® Dark Blue, MERCK®, MICA, TITANIUM DIOXIDE, FERRIC FERROCYANIDE

### COLORONA® Chameleon, MERCK®, CI 77491 (IRON OXIDES), MICA

### COLORONA® Aborigine Amber, MERCK®, MICA, CI 77499 (IRON OXIDES), CI 77891 (TITANIUM DIOXIDE)

### COLORONA® Blackstar Blue, MERCK®, CI 77499 (IRON OXIDES), MICA

### COLORONA® Patagonian Purple, MERCK®, MICA, CI 77491 (IRON OXIDES), CI 77891 (TITANIUM DIOXIDE), CI 77510 (FERRIC FERROCYANIDE)

### COLORONA® Red Brown, MERCK®, MICA, CI 77491 (IRON OXIDES), CI 77891 (TITANIUM DIOXIDE)

### COLORONA® Russet, MERCK®, CI 77491 (TITANIUM DIOXIDE), MICA, CI 77891 (IRON OXIDES)

### COLORONA® Imperial Red, MERCK®, MICA, TITANIUM DIOXIDE (CI 77891), D&C RED NO. 30 (CI 73360)

### COLORONA® Majestic Green, MERCK®, CI 77891 (TITANIUM DIOXIDE), MICA, CI 77288 (CHROMIUM OXIDE GREENS)

### COLORONA® Light Blue, MERCK®, MICA, TITANIUM DIOXIDE (CI 77891), FERRIC FERROCYANIDE (CI 77510)

### COLORONA® Red Gold, MERCK®, MICA, CI 77891 (TITANIUM DIOXIDE), CI 77491 (IRON OXIDES)

### COLORONA® Gold Plus MP 25, MERCK®, MICA, TITANIUM DIOXIDE (CI 77891), IRON OXIDES (CI 77491)

### COLORONA® Carmine Red, MERCK®, MICA, TITANIUM DIOXIDE, CARMINE

### COLORONA® Blackstar Green, MERCK®, MICA, CI 77499 (IRON OXIDES)

### COLORONA® Bordeaux, MERCK®, MICA, CI 77491 (IRON OXIDES)

### COLORONA® Bronze, MERCK®, MICA, CI 77491 (IRON OXIDES)

### COLORONA® Bronze Fine, MERCK®, MICA, CI 77491 (IRON OXIDES)

### COLORONA® Fine Gold MP 20, MERCK®, MICA, CI 77891 (TITANIUM DIOXIDE), CI 77491 (IRON OXIDES)

### COLORONA® Sienna Fine, MERCK®, CI 77491 (IRON OXIDES), MICA

### COLORONA® Sienna, MERCK®, MICA, CI 77491 (IRON OXIDES)

COLORONA® Precious Gold, MERCK®, Mica, CI 77891 (Titanium dioxide), Silica, CI 77491 (Iron oxides), Tin oxide

### COLORONA® Sun Gold Sparkle MP 29, MERCK®, MICA, TITANIUM DIOXIDE, IRON OXIDES, MICA, CI 77891, CI 77491 (EU)

COLORONA® Mica Black, MERCK®, CI 77499 (Iron oxides), Mica, CI 77891 (Titanium dioxide)
COLORONA® Bright Gold, MERCK®, Mica, CI 77891 (Titanium dioxide), CI 77491 (Iron oxides)

### COLORONA® Blackstar Gold, MERCK®, MICA, CI 77499 (IRON OXIDES)

Other particularly preferred color pigments with the trade name XIRONA® are for example:

### XIRONA® Golden Sky, MERCK®, Silica, CI 77891 (Titanium Dioxide), Tin Oxide

### XIRONA® Caribbean Blue, MERCK®, Mica, CI 77891 (Titanium Dioxide), Silica, Tin Oxide

### XIRONA® Kiwi Rose, MERCK®, Silica, CI 77891 (Titanium Dioxide), Tin Oxide

### XIRONA® Magic Mauve, MERCK®, Silica, CI 77891 (Titanium Dioxide), Tin Oxide.

In addition, particularly preferred color pigments with the trade name UNIPURE® are for example:

### UNIPURE® Red LC 381 EM, SENSIENT® CI 77491 (Iron Oxides), Silica

### UNIPURE® Black LC 989 EM, SENSIENT®, CI 77499 (Iron Oxides), Silica

### UNIPURE® Yellow LC 182 EM, SENSIENT®, CI 77492 (Iron Oxides), Silica

In a further version, the composition or preparation according to the present disclosure may also comprise one or more coloring compounds selected from the group of organic pigments

The organic pigments according to the present disclosure are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyrrole, indigo, thioindigo, dioxazine and/or triarylmethane compounds.

Particularly suitable organic pigments are, for example, carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred version, a process according to the present disclosure is exemplified in that the composition (B) and/or the composition (C) comprises at least one colorant compound from the group of organic pigments selected from the group of carmine, quinacridone, phthalocyanine, sorghum, blue pigments having the color index numbers Cl 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

Furthermore, the organic pigment may also be a colored lacquer. In the sense of the present disclosure, the term color lacquer means particles comprising a layer of absorbed dyes, the unit of particle and dye being insoluble under the above mentioned conditions. The particles can, for example, be inorganic substrates, which can be aluminum, silica, calcium borosilicate, calcium aluminum borosilicate or even aluminum.

For example, alizarin color varnish can be used.

Due to their excellent resistance to light and temperature, the use of the aforementioned pigments according to the present disclosure is particularly preferred. It is also preferred if the pigments used have a certain particle size. This particle size leads on the one hand to an even distribution of the pigments in the formed polymer film and on the other hand avoids a rough hair or skin feeling after application of the cosmetic product. According to the present disclosure, it is therefore advantageous if the at least one pigment has an average particle size D₅₀ of about 1.0 to about 50 μm, preferably about 5.0 to about 45 μm, preferably about 10 to about 40 μm, in particular about 14 to about 30 μm. The mean particle size D₅₀, for example, can be determined using dynamic light scattering (DLS).

The pigment or pigments may be used in an amount of from about 0.001 to about 20% by weight, in particular from about 0.05 to about 5% by weight, in each case based on the total weight of the composition or preparation according to the present disclosure.

As colorant compounds, the compositions according to the present disclosure may also comprise one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments. Preferably, the direct dyes in the sense of the present disclosure have a solubility in water (760 mmHg) at 25° C. of more than 1.0 g/L. More preferably, the direct dyes in the sense of the present disclosure have a solubility in water (760 mmHg) at 25° C. of more than 1.5 g/L.

Direct dyes can be divided into anionic, cationic and non-ionic direct dyes.

In a further preferred version, an agent according to the present disclosure is exemplified in that it comprises at least one anionic, cationic and/or nonionic direct dye as the coloring compound.

In a further preferred version, a process according to the present disclosure is exemplified in that the composition (B) and/or the composition (C) comprises at least one colorant compound selected from the group of anionic, nonionic, and/or cationic direct dyes.

Suitable cationic direct dyes include Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes which have at least one carboxylic acid grouping (—COOH) and/or one sulfonic acid grouping (—SO₃H). Depending on the pH, the protonated forms (—COOH, —SO₃H) of the carboxylic or sulfonic acid groups are in equilibrium with their deprotonated forms (—COO—, —SO₃— present). As the pH decreases, the proportion of protonated forms increases. If direct dyes are used in the form of their salts, the carboxylic acid groups or sulphonic acid groups are present in deprotonated form and are neutralized with corresponding stoichiometric equivalents of cations to maintain electro neutrality. Acid dyes according to the present disclosure can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L.

The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential feature of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group may be selected as particularly suitable acid dyes: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C. 29, Covacap Jaune W 1100 (LCW), Sicovit Tartrazine 85 E 102 (BASF®), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C-Rot 46, Real Red D, FD&C Red Nr. 2, Food Red 9, Naphthol Red S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido Blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I. 42100), Acid Green 22 (C.I. 42170), Acid Green 25 (CI 61570, Japan Green 201, D&C Green No. 5), Acid Green 50 (Acid Brilliant Green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. A stir-fish is added. Then add 100 ml of water. This mixture is heated to 25° C. on a magnetic stirrer while stirring. It is stirred for 60 minutes. The aqueous mixture is then visually assessed. If there are still undissolved residues, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).

Acid Yellow 3 is a mixture of the sodium salts of mono- and disulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)-1,3-naphthalene disulfonate and has a very high water solubility of more than 20% by weight.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a solubility in water of more than 20% by weight (25° C.).

Furthermore, thermochromic dyes can also be used. Thermochromism is the property of a material to change its color reversibly or irreversibly depending on the temperature. This can be done by changing the intensity and/or the wavelength maximum.

Finally, it is also possible to use photochromic dyes. Photochromism involves the property of a material to change its color reversibly or irreversibly depending on the irradiation with light, especially UV light. This can be done by changing the intensity and/or the wavelength maximum.

### Film Forming Polymers

The preparations described above, in particular preparations (B), (C) and (D), highly preferred, preparation (D), may comprise at least one film-forming polymer.

Polymers are macromolecules with a molecular weight of at least about 1000 g/mol, preferably of at least about 2500 g/mol, particularly preferably of at least about 5000 g/mol, which include identical, repeating organic units. The polymers of the present disclosure may be synthetically produced polymers which are manufactured by polymerization of one type of monomer or by polymerization of different types of monomers which are structurally different from each other. If the polymer is produced by polymerizing a type of monomer, it is called a homo-polymer. If structurally different monomer types are used in polymerization, the resulting polymer is called a copolymer.

The maximum molecular weight of the polymer depends on the degree of polymerization (number of polymerized monomers) and the batch size and is determined by the polymerization method. For the purposes of the present disclosure, it is preferred that the maximum molecular weight of the film-forming hydrophobic polymer (c) is not more than about 107 g/mol, preferably not more than about 106 g/mol and particularly preferably not more than about 105 g/mol.

In the sense of the present disclosure, a film-forming polymer is a polymer which is capable of forming a film on a substrate, for example on a keratinous material or a keratinous fiber. The formation of a film can be demonstrated, for example, by looking at the keratin material treated with the polymer under a microscope.

The film-forming polymers can be hydrophilic or hydrophobic.

In a first version, it may be preferred to use at least one hydrophobic film-forming polymer in preparation (B), (C) and/or (D), especially in preparation (D).

A hydrophobic polymer is defined as a polymer that has a solubility in water at 25° C. (760 mmHg) of less than 1% by weight.

The water solubility of the film-forming, hydrophobic polymer can be determined in the following way, for example. 1.0 g of the polymer is placed in a beaker. Make up to 100 g with water. A stir-fish is added and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1% by weight.

These include acrylic acid-type polymers, polyurethanes, polyesters, polyamides, polyureas, cellulose polymers, nitrocellulose polymers, silicone polymers, acrylamide-type polymers and polyisoprenes.

Particularly well suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

In a further preferred version, a composition according to the present disclosure is exemplified in that it comprises at least one film-forming, hydrophobic polymer (c) which is selected from the group of the copolymers of acrylic acid, the copolymers of methacrylic acid, the homopolymers or copolymers of acrylic acid esters, the homopolymers or copolymers of methacrylic acid esters homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

Film-forming hydrophobic polymers selected from the group of synthetic polymers, polymers obtainable by free-radical polymerization or natural polymers have proved to be particularly suitable for solving the problem according to the present disclosure.

Other particularly well-suited film-forming hydrophobic polymers may be selected from the homopolymers or copolymers of olefins, such as cycloolefins, butadiene, isoprene or styrene, vinyl ethers, vinyl amides, the esters or amides of (meth)acrylic acid having at least one C₁-C₂₀ alkyl group, an aryl group or a C₂-C₁₀ hydroxyalkyl group.

Further film forming hydrophobic polymers may be selected from the homo- or copolymers of isooctyl (meth)acrylate; isononyl (meth)acrylate; 2-ethylhexyl (meth)acrylate; lauryl (meth)acrylate); isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate; and/or mixtures thereof.

Further film-forming hydrophobic polymers may be selected from the homo- or copolymers of (meth)acrylamide; N-alkyl-(meth)acrylamides, in particular those containing C₂-C₁₈ alkyl groups, such as N-ethyl-acrylamide, N-tert-butyl-acrylamide, le N-octylacrylamide; N-di(C₁-C₄)alkyl-(meth)acrylamide.

Other preferred anionic copolymers are, for example, copolymers of acrylic acid, methacrylic acid or their C₁-C₆ alkyl esters, as sold under the INCI declaration Acrylates Copolymers. A suitable commercial product is, for example, ACULYN® 33 from Rohm & Haas. However, copolymers of acrylic acid, methacrylic acid or their C₁-C₆ alkyl esters and the esters of an ethylenically unsaturated acid and an alkoxylated fatty alcohol are also preferred. Suitable ethylenically unsaturated acids are especially acrylic acid, methacrylic acid and itaconic acid; suitable alkoxylated fatty alcohols are especially steareth-20 or ceteth-20.

Very particularly preferred polymers on the market are, for example, ACULYN® 22 (Acrylates/Steareth-20 Methacrylate Copolymer), ACULY® 28 (Acrylates/Beheneth-25 Methacrylate Copolymer), STRUCTURE® 2001 (Acrylates/Steareth-20 Itaconate Copolymer), STRUCTURE® 3001 (Acrylates/Ceteth-20 Itaconate Copolymer), STRUCTURE® Plus (Acrylates/Aminoacrylates C10-30 Alkyl PEG-20 Itaconate Copolymer), CARBOPOL® 1342, 1382, Ultrez 20, Ultrez 21 (Acrylates/C10-30 Alkyl Acrylate Crosspolymer), SYNTHALEN® W 2000 (Acrylates/Palmeth-25 Acrylate Copolymer) or Soltex OPT (Acrylates/C₁₂-22 Alkyl methacrylate Copolymer) distributed Rohme and Haas.

Suitable polymers based on vinyl monomers may include, for example, the homopolymers and copolymers of N-vinylpyrrolidone, vinylcaprolactam, vinyl-(C₁-C₆)alkyl-pyrrole, vinyl-oxazole, vinyl-thiazole, vinylpyrimidine, vinylimidazole.

Furthermore, the copolymers octylacrylamide/acrylates/butylaminoethyl-methacrylate copolymer, as commercially marketed under the trade names AMPHOMER® or LOVOCRYL® 47 by NATIONAL STARCH, or the copolymers of acrylates/octylacrylamides marketed under the trade names DERMACRYL® LT and DERMACRYL® 79 by NATIONAL STARCH are particularly suitable.

Suitable polymers based on olefins may include, for example, the homopolymers and copolymers of ethylene, propylene, butene, isoprene and butadiene.

In another version, block copolymers can be used as film-forming hydrophobic polymers, which comprise at least one block of styrene or the derivatives of styrene. These block copolymers can be copolymers that contain one or more other blocks in addition to a styrene block, such as styrene/ethylene, styrene/ethylene/butylene, styrene/butylene, styrene/isoprene, styrene/butadiene. Such polymers are commercially distributed by BASF® under the trade name “Luvitol HSB”.

It was also possible to obtain intense and washfast staining when the preparation (B), (C) and/or (D), particularly in the preparation (D), contained at least one film-forming polymer selected from the group of the homopolymers and copolymers of acrylic acid, the homopolymers and copolymers of methacrylic acid, the homopolymers and copolymers of acrylic acid esters, the homopolymers and copolymers of methacrylic acid esters, the homopolymers and copolymers of acrylic acid amides homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

In a further preferred version, a method according to the present disclosure is exemplified in that the preparation (B), (C) and/or (D), most particularly the preparation (D), contains at least one film-forming polymer selected from the group of homopolymers and copolymers of acrylic acid, homopolymers and copolymers of methacrylic acid, homopolymers and copolymers of acrylic acid esters, homopolymers and copolymers of methacrylic acid esters, homopolymers and copolymers of acrylic acid amides homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

In a first version, it may be preferred to use at least one hydrophilic film-forming polymer in preparation (B), (C) and/or (D), especially in preparation (D).

A hydrophilic polymer is defined as a polymer having a solubility in water at 25° C. (760 mmHg) of more than 1% by weight, preferably more than 2% by weight.

The water solubility of the film-forming, hydrophilic polymer can be determined in the following way, for example. 1.0 g of the polymer is placed in a beaker. Make up to 100 g with water. A stir-fish is added and the mixture is heated to 25° C. on a magnetic stirrer while stirring. It is stirred for 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, the solubility of the polymer is more than 1% by weight.

Nonionic, anionic and cationic polymers can be used as film-forming, hydrophilic polymers.

Suitable film-forming hydrophilic polymers can be selected, for example, from the group of polyvinylpyrrolidone (co)polymers, polyvinyl alcohol (co)polymers, vinyl acetate (co)polymers, carboxyvinyl (co)polymers, acrylic acid (co)polymers, methacrylic acid (co)polymers, natural gums, polysaccharides and/or acrylamide (co)polymers.

Furthermore, it is particularly preferred to use polyvinylpyrrolidone (PVP) and/or a vinylpyrrolidone-containing copolymer as the film-forming hydrophilic polymer.

In another particularly preferred version, an agent according to the present disclosure is exemplified in that it contains (c) at least one film-forming, hydrophilic polymer selected from the group of polyvinylpyrrolidone (PVP) and the copolymers of polyvinylpyrrolidone.

It is further preferred if the agent according to the present disclosure comprises polyvinylpyrrolidone (PVP) as the film-forming hydrophilic polymer. Surprisingly, the wash fastness of the colorations obtained with agents containing PVP (b9 was also very good.

Particularly well suited polyvinylpyrrolidones are, for example, available under the name LUVISKOL® K from BASF® SE, especially LUVISKOL® K 90 or LUVISKOL® K 85 from BASF® SE.

The polymer PVP K30, which is marketed by ASHLAND® (ISP, POI Chemical), can also be used as another explicitly very well suited polyvinylpyrrolidone (PVP). PVP K 30 is a polyvinylpyrrolidone which is highly soluble in cold water and has the CAS number 9003-39-8. The molecular weight of PVP K 30 is about 40000 g/mol.

Other particularly suitable polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115 and available from BASF®.

The use of film-forming hydrophilic polymers from the group of copolymers of polyvinylpyrrolidone has also led to particularly good and washfast color results.

Vinylpyrrolidone-vinyl ester copolymers, such as those marketed under the trademark LUVISKOL® (BASF®), are particularly suitable film-forming hydrophilic polymers. LUVISKOL® VA 64 and LUVISKOL® VA 73, both vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are particularly preferred in cosmetic compositions.

Vinylpyrrolidone-vinyl acetate copolymers are marketed by BASF® SE under the name LUVISKOL® VA. For example, a VP/Vinyl Caprolactam/DMAPA Acrylates copolymer is sold under the trade name AQUAFLEX® SF-40 by ASHLAND® Inc. For example, a VP/DMAPA acrylates copolymer is marketed by ASHLAND® under the name STYLEZE® CC-10 and is a highly preferred vinylpyrrolidone-containing copolymer.

Other suitable copolymers of polyvinylpyrrolidone may also be those obtained by reacting N-vinylpyrrolidone with at least one further monomer from the group of V-vinylformamide, vinyl acetate, ethylene, propylene, acrylamide, vinylcaprolactam, vinylcaprolactone and/or vinyl alcohol.

In another particularly preferred version, an agent according to the present disclosure is exemplified in that it comprises at least one film-forming hydrophilic polymer selected from the group of polyvinylpyrrolidone (PVP), vinylpyrrolidone/vinyl acetate copolymers, vinylpyrrolidone/styrene copolymers, vinylpyrrolidone/ethylene copolymers, vinylpyrrolidone/propylene copolymers, vinylpyrrolidone/vinylcaprolactam copolymers, vinylpyrrolidone/vinylformamide copolymers and/or vinylpyrrolidone/vinyl alcohol copolymers.

Another useful copolymer of vinylpyrrolidone is the polymer known by the INCI name maltodextrin/VP copolymer.

Furthermore, intensively dyed keratin material, especially hair, with very good washfastness could be obtained if a non-ionic, film-forming, hydrophilic polymer was used as the film-forming, hydrophilic polymer.

In a first version, it may be preferred if preparation (B), (C) and/or (D), in particular preparation (D), comprise at least one non-ionic, film-forming, hydrophilic polymer.

According to the present disclosure, a non-ionic polymer is understood to be a polymer which in a protic solvent—such as water—under standard conditions does not carry structural units with permanent cationic or anionic groups, which must be compensated by counterions while maintaining electron neutrality. Cationic groups include, for example, quaternized ammonium groups but not protonated amines. Anionic groups include carboxylic and sulphonic acid groups.

Particular preference is given to products containing, as a non-ionic, film-forming, hydrophilic polymer, at least one polymer selected from the group of
Polyvinylpyrrolidone,
Copolymers of N-vinylpyrrolidone and vinyl esters of carboxylic acids having 2 to about 18 carbon atoms, in particular of N-vinylpyrrolidone and vinyl acetate,
Copolymers of N-vinylpyrrolidone and N-vinylimidazole and methacrylamide,
Copolymers of N-vinylpyrrolidone and N-vinylimidazole and acrylamide,
Copolymers of N-vinylpyrrolidone with N,N-di(C1 to C4)-alkylamino-(C2 to C4)-alkylacrylamide,

If copolymers of N-vinylpyrrolidone and vinyl acetate are used, it is again preferable if the molar ratio of the structural units contained in the monomer N-vinylpyrrolidone to the structural units of the polymer contained in the monomer vinyl acetate is in the range from about 20:80 to about 80:20, in particular from about 30:70 to about 60:40. Suitable copolymers of vinylpyrrolidone and vinyl acetate are available, for example, under the trademarks LUVISKOL® VA 37, LUVISKOL® VA 55, LUVISKOL® VA 64 and LUVISKOL® VA 73 from BASF® SE.

Another particularly preferred polymer is selected from the INCI designation VP/Methacrylamide/Vinyl Imidazole Copolymer, which is available under the trade name LUVISET® Clear from BASF® SE.

Another particularly preferred non-ionic, film-forming, hydrophilic polymer is a copolymer of N-vinylpyrrolidone and N,N-dimethylaminiopropylmethacrylamide, which is sold under the INCI designation VP/DMAPA Acrylates Copolymer e.g. under the trade name STYLEZE® CC 10 by ISP.

A cationic polymer according to the present disclosure is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI designation: polyquaternium-69), which is marketed, for example, under the trade name AQUASTYLE® 300 (28-32% by weight of active substance in ethanol-water mixture, molecular weight 350000) by ISP.

Other suitable film-forming, hydrophilic polymers include
Vinylpyrrolidone-vinylimidazolium methochloride copolymers, as offered under the designations LUVIQUAT® FC 370, FC 550 and the INCI designation Polyquatemium-16 as well as FC 905 and HM 552,
Vinylpyrrolidone-vinylcaprolactam-acrylate terpolymers, as they are commercially available with acrylic acid esters and acrylic acid amides as a third monomer component, for example under the name AQUAFLEX® SF 40.

Polyquaternium-11 is the reaction product of diethyl sulphate with a copolymer of vinyl pyrrolidone and dimethylaminoethyl methacrylate. Suitable commercial products are available under the names DEHYQUART® CC 11 and LUVIQUAT® PQ 11 PN from BASF® SE or GAFQUAT® 440, GAFQUAT® 734, GAFQUAT® 755 or GAFQUAT® 755N from ASHLAND® Inc.

Polyquaternium-46 is the reaction product of vinylcaprolactam and vinylpyrrolidone with methylvinylimidazolium methosulfate and is available for example under the name LUVIQUAT® Hold from BASF® SE. Polyquaternium-46 is preferably used in an amount of about 1 to about 5% by weight—based on the total weight of the cosmetic composition. It particularly prefers to use polyquaternium-46 in combination with a cationic guar compound. It is even highly preferred that polyquaternium-46 is used in combination with a cationic guar compound and polyquatemium-11.

Suitable anionic film-forming, hydrophilic polymers can be, for example, acrylic acid polymers, which can be in non-crosslinked or crosslinked form. Such products are sold commercially under the trade names CARBOPOL® 980, 981, 954, 2984 and 5984 by Lubrizol or under the names SYNTHALEN® M and SYNTHALEN® K by 3V Sigma (The Sun Chemicals, Inter Harz).

Examples of suitable film-forming hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Suitable film-forming, hydrophilic polymers from the group of acrylamides are, for example, polymers which are produced from monomers of (methyl)acrylamido-C₁-C₄-alkyl sulphonic acid or the salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Preferred polymers of poly(meth)arylamido-C₁-C₄-alkyl sulfonic acids are crosslinked and at least about 90% neutralized. These polymers can or cannot be cross-linked.

Cross-linked and totally or partially neutralized polymers of the poly-2-acrylamido-2-methylpropane sulphonic acid type are known under the INCI designation “Ammonium Polyacrylamido-2-methylpropanesulphonates” or “Ammonium Polyacryldimethyltauramides”.

Another preferred polymer of this type is the cross-linked poly-2-acrylamido-2-methyl-propanesulphonic acid polymer marketed by Clamant under the trade name Hostacerin AMPS, which is partially neutralized with ammonia.

In a further explicitly highly preferred version, a process according to the present disclosure is exemplified in that the preparation (B), (C) and/or (D), particularly the preparation (D), comprises at least one anionic, film-forming, polymer.

In this context, the best results were obtained when preparation (B), (C) and/or (D), and more particularly preparation (D), contains at least one film-forming polymer comprising at least one structural unit of formula (P-I) and at least one structural unit of formula (P-II).

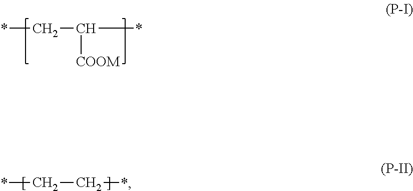

where

M represents a hydrogen atom or ammonium (NH₄), sodium, potassium, ½ magnesium or ½ calcium.

In a further preferred version, a method according to the present disclosure is exemplified in that the preparation (B), (C) and/or (D), most particularly the preparation (D), includes at least one film-forming polymer comprising at least one structural unit of the formula (P-I) and at least one structural unit of the formula (P-II)

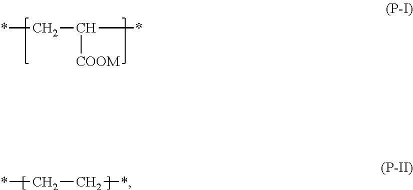

where
M represents a hydrogen atom or ammonium (NH₄), sodium, potassium, ½ magnesium or ½ calcium.

When M represents a hydrogen atom, the structural unit of the formula (P-I) is based on an acrylic acid unit.

When M is an ammonium counterion, the structural unit of the formula (P-I) is based on the ammonium salt of acrylic acid.
When M represents a sodium counterion, the structural unit of the formula (P-I) is based on the sodium salt of acrylic acid.
When M represents a potassium counterion, the structural unit of the formula (P-I) is based on the potassium salt of acrylic acid.
When M is a half equivalent of a magnesium counterion, the structural unit of the formula (P-I) is based on the magnesium salt of acrylic acid.
When M represents half an equivalent of a calcium counterion, the structural unit of the formula (P-I) is based on the calcium salt of acrylic acid.

The film-forming polymer(s) according to the present disclosure is/are preferably used in certain ranges of amounts in the preparations (B), (C) and/or (D) according to the present disclosure. In this context, it has been shown to be particularly preferred for solving the problem according to the present disclosure if the preparation contains—in each case based on its total weight—one or more film-forming polymers in a total amount of from about 0.1 to about 18.0% by weight, preferably from about 1.0 to about 16.0% by weight, more preferably from about 5.0 to about 14.5% by weight and highly preferably from about 8.0 to about 12.0% by weight.

In a further preferred version, a process according to the present disclosure is exemplified in that the preparation (B), (C) and/or (D) contains—based on their respective total weight—one or more film-forming polymers in a total amount of from about 0.1 to about 18.0% by weight, preferably from about 1.0 to about 16.0% by weight, more preferably from about 5.0 to about 14.5% by weight and highly preferably from about 8.0 to about 12.0% by weight.

### Multi-Component Packaging Unit (Kit-of-Parts)

To increase user convenience, all preparations necessary for the application process, in particular for the dyeing process, are provided to the user in the form of a multi-component packaging unit (kit-of-parts).

A second subject of the present disclosure is therefore a multi-component packaging unit (kit-of-parts) for treating keratinous material, comprehensively packaged separately from one another.
a first container comprising a first composition (A) and
a second container comprising a second composition (B), wherein compositions (A) and (B) having already been disclosed in detail in the description of the first subject matter of the present disclosure.

Furthermore, the multi-component packaging unit according to the present disclosure may further comprise a third packaging unit containing a cosmetic preparation (C). The preparation (C) contains, as described above, particularly preferably at least one color-imparting compound.

In a highly preferred version, the multi-component packaging unit (kit-of-parts) according to the present disclosure comprises separately assembled
a third container comprising a third composition (C), the third composition (C) having already been disclosed in detail in the description of the first subject matter of the present disclosure.

Furthermore, the multi-component packaging unit according to the present disclosure may further comprise a fourth packaging unit containing a cosmetic preparation (D). The preparation (D) contains, as described above, particularly preferably at least one film-forming polymer.

In a highly preferred version, the multi-component packaging unit (kit-of-parts) according to the present disclosure comprises separately assembled
a fourth container comprising a fourth composition (D), the fourth composition (D) having already been disclosed in detail in the description of the first subject matter of the present disclosure.

With respect to the other preferred versions of the multi-component packaging unit according to the present disclosure, the same applies mutatis mutandis to the procedure according to the present disclosure.

### Examples

### 1 Preparation of the Silane Blend (Composition (A))

A reactor with heatable/coolable outer shell and with a capacity of 10 liters was filled with 4.67 kg of methyltrimethoxysilane (34.283 mol). With stirring, 1.33 kg of (3-aminopropyl)triethoxysilane (6.008 mol) was then added. This mixture was stirred at 30° C. Subsequently, 670 ml of distilled water (37.18 mol) was added dropwise with vigorous stirring while maintaining the temperature of the reaction mixture at 30° C. under external cooling. After completion of the water addition, stirring was continued for another 10 minutes. A vacuum of 280 mbar was then applied and the reaction mixture heated to a temperature of 44° C. Once the reaction mixture reached the temperature of 44° C., the ethanol and methanol released during the reaction were distilled off over a period of 190 minutes. In the course of distillation, the vacuum was lowered to 200 mbar. The distilled alcohols were collected in a cooled receiver. The reaction mixture was then allowed to cool to room temperature. To the mixture thus obtained, 3.33 kg of hexamethyldisiloxane was then dropped with stirring. It was stirred for 10 minutes. In each case, 100 ml of the silane blend was filled into a bottle with a capacity of 100 ml and screw cap with seal. After filling, the bottles were tightly sealed. The water content was less than 2.0% by weight.

### 2 Preparation of the Composition (B)

The following compositions (B) were prepared (unless otherwise stated, all figures are in % by weight).

### Composition (B)

| | | B-E1 |
|---|---|---|
| | B-V1 | Emulsion |
| | Gel | Present |
| | Comparison | disclosure |
| Hydroxyethyl cellulose | 1.0 | — |
| Easynov (Octyldodecanol & Octyldodecyl | — | 10.0 |
| Xyloside & PEG-30 Dipolyhydroxystearate, | | |
| SEPPIC) | | |
| 1.2-propanediol | — | 7.0 |
| Water (distilled) | ad 100 | ad 100 |

### 3 Preparation of Compositions (C) and (D)

The following compositions were prepared (unless otherwise stated, all figures are in % by weight).

### Composition (C)

| | % in weight |
|---|---|
| Lavanya Belmont | 35.0 |
| Phthalocyanine blue pigment CI 74160 | |
| PEG-12 Dimethicone | ad 100 |

### Composition (D)

| | % in weight |
|---|---|
| Ethylene/Sodium Acrylate | 40.0 |
| Copolymer (25% solution) | |
| Water | ad 100 |

### 5. Application

The ready-to-use composition was prepared by mixing 1.5 g of the composition (A), 20.0 g of the composition (B) and 1.5 g of the composition (C), respectively. Compositions (A), (B) and (C) were shaken for 1 minute each. Then this ready-to-use agent was dyed on two strands of hair (Kerling, Euronatural hair white) each.

Three minutes after completion of shaking, the ready-to-use composition was applied to a first strand (strand 1), left to act for 1 min, and then rinsed out. 10 min after completion of shaking, the ready-to-use composition was applied to a second strand (strand 2), left to act for 1 min, and then rinsed out.

Subsequently, the composition (D) was applied to each strand of hair, left to act for 1 minute and then also rinsed with water.

The two dyed strands were each dried and visually compared under a daylight lamp.

| | | |
|---|---|---|
| Step one: | (A) + (B-V1) + (C) | (A) + (B-E1) + (C) |
| Step two: | D | D |
| Color difference | high | low |
| between strand 1 and 2 | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for treating keratinous material, comprising:
applying a first composition (A) to the keratinous material, the first composition (A) comprising, relative to the total weight of the composition (A)
(A1) less than about 10% by weight of water and
(A2) one or more organic C₁-C₆ alkoxy silanes and/or their condensation products, and
applying a second composition (B) to the keratinous material, the second composition (B) comprising
(B1) Water,
(B2) at least a first surfactant, and
(B3) at least a second surfactant which is structurally different from the first surfactant (B2).

2. (canceled)

3. The method according to claim 1, wherein the one or more organic C₁-C₆ alkoxy silanes (A2) comprise a composition of formula (S-I) and/or (S-II),
R₁R₂N-L-Si(OR₃)ₐ(R₄)_{b} (S-I)
where
R₁, R₂ independently represent a hydrogen atom or a C₁-C₆ alkyl group,
L is a linear or branched divalent C₁-C₂₀ alkylene group,
R₃, R₄ independently represent a C₁-C₆ alkyl group,
a, stands for an integer from 1 to 3, and
b is the integer 3-a, and
(R₅O)_{c}(R₆)_{d}Si-(A)ₑ-[NR₇-(A′)]_{f}-[O-(A′″)]_{g}—[NR₈-(A″″)]ₕ-Si(R_{6′})_{d′}(OR_{5′})_{c′} (S-II),
where
R₅, R_{5′}, R_{5″}, R₆, R_{6′}, and R_{6″} independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (S-III),
-(A″″)-Si(R_{6″})_{d″}(OR_{5″})_{c″} (S-III),
wherein
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0,
and/or their condensation products.

4. The method according to claim 3, wherein the first composition (A) comprises the at least one C₁-C₆ organic alkoxysilane (A2) of formula (S-I) chosen from the group of
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-Dimethylaminoethyl)triethoxysilane,
(2-Dimethylaminoethyl)trimethoxysilane
and/or their condensation products.

5. The method according to claim 3, wherein the first composition (A) comprises the one or more organic C₁-C₆ alkoxy silanes (A2) of formula (S-IV),
R₉Si(OR₁₀)ₖ(R₁₁)ₘ (S-IV),
where
R₉ represents a C₁-C₁₂ alkyl group,
R₁₀ stands for a C₁-C₆ alkyl group,
R₁₁ stands for a C₁-C₆ alkyl group
k is an integer from 1 to 3, and
m stands for the integer 3-k.
and/or their condensation products.

6. The method according to claim 3, wherein the first composition (A) comprises the at least one C₁-C₆ organic alkoxysilane (A2) of formula (S-I) chosen from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
combinations thereof,
and/or their condensation products.

7. (canceled)

8. The method according to claim 1, wherein the first composition (A) comprises at least one cosmetic ingredient chosen from the group of hexamethyldisiloxane. octamethyltrisiloxane, decamethyltetrasiloxane, hexamethylcyclotrisiloxane, octamethylcyclotetrasiloxane and decamethylcyclopentasiloxane

9. The method according to claim 1, wherein the first composition (A) comprises—based on the total weight of the composition (A)—10.0 to 50.0% by weight of hexamethyldisiloxane.

10. (canceled)

11. (canceled)

12. (canceled)

13. The method according to claim 1, wherein the second composition (B) comprises
at least one first nonionic surfactant (B2), and
at least one second nonionic surfactant (B3) which is structurally different from the first nonionic surfactant (B2).

14. The method according to claim 1, wherein the second composition (B) comprises at least one first nonionic surfactant (B2) of formula (T-I), 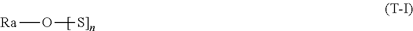
wherein
Ra represents a saturated or unsaturated, unbranched or branched C₁₂-C₃₀ alkyl group, and
n represents an integer from 1 to about 10, and
S represents a sugar residue with 5 or 6 carbon atoms.

15. The method according to claim 14, wherein the second composition (B) comprises the at least one first nonionic surfactant (B2) of formula (T-I) in which
Ra represents a saturated, branched C₁₂-C₃₀ alkyl group,
n represents for the number 1 and
S represents for a xylitol residue.

16. (canceled)

17. The method according to claim 1, wherein the second composition (B) comprises at least one second nonionic surfactant (B3) of formula (T-II), 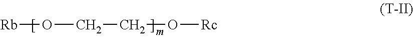
wherein
Rb, Rc independently of one another represent a saturated, unbranched or branched, unsubstituted or substituted, C₁₂-C₃₀-alkanoyl group, and
m represents an integer from 1 to about 60.

18. (canceled)

19. The method according to claim 1, wherein the second composition (B) comprises one or more fat components selected from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

20. (canceled)

21. The method according to claim 1, further comprising:
mixing the first compostions (A) with the second composition (B) prior to applying the first composition (A) to the keratinous material, and prior to applying the second composition (B) to the keratinous material.

22. The method according to claim 1, further comprising:
applying a third composition (C) to the keratinous material, the third composition (C) comprising;
at least one coloring compound selected from the group of pigments and/or direct dyes.

23. The method according to claim 22, further comprising: mixing the first composition (A) with the second composition (B) and the third composition (C) prior to applying the first composition (A) to the keratinous material, prior to applying the second composition (B) to the keratinous material, and prior to applying the third composition (C) to the keratinous material.

24. (canceled)

25. The method according to claim 1, further comprising:
applying a fourth composition (D) to the keratinous material, the fourth composition (D) comprising
at least one film-forming polymer,

26. The method according to claim 22, wherein the composition (B) and/or the composition (C) comprise at least one coloring compound chosen from the group of inorganic pigments chosen from the group of colored metal oxides, metal hydroxides, metal oxide hydrates, silicates, metal sulfides, complex metal cyanides, metal sulphates, bronze pigments and/or colored mica- or mica-based pigments coated with at least one metal oxide and/or a metal oxychloride.

27. (canceled)

28. (canceled)

29. A kit-of-parts for treating keratinous material, comprising separately packaged
a first container containing a first composition (A) and
a second container containing a second composition (B), wherein
the first composition (A) comprises;
(A1) less than about 10% by weight of water and
(A2) one or more organic C₁-C₆ alkoxy silanes and/or their condensation products;
and the second composition (B) comprises;
(B1) Water,
(B2) at least a first surfactant, and
(B3) at least a second surfactant which is structurally different from the first surfactant (B2).

30. The kit-of-parts according to claim 29, further comprising separately packaged
a third container containing a third composition (C), wherein the third composition (C) comprises at least one coloring compound selected from the group of pigments and/or direct dyes.

31. The kit-of-parts according to claim 29, further comprising separately packaged
a fourth container containing a fourth composition (D), the fourth composition (D) comprising at least one film-forming polymer.
